(19) United States
(12) Patent Application Publication
(10) Pub. No.: US 2025/0128093 A1
(43) Pub. Date: Apr. 24, 2025

(54) Charged Particle Therapy System Utilizing Fluidically Coupled Chambers for Energy Selection

(71) Applicant: Mayo Foundation for Medical Education and Research, Rochester, MN (US)

(72) Inventors: Chris J. Beltran, Ponte Vedra Beach, FL (US); Keith M. Furutani, Rochester, MN (US)

(21) Appl. No.: 18/834,095

(22) PCT Filed: Dec. 5, 2022
(86) PCT No.: PCT/US2022/080896

§ 371 Date: Jul. 29, 2024

Related U.S. Application Data
(60) Provisional application No. 63/304,346, filed on Jan. 28, 2022.

(51) Int. Cl. A61N 5/10

(52) U.S. Cl. CPC A61N 5/1043; A61N 5/1081; A61N 2005/1087; A61N 2005/1095

(57) ABSTRACT

Compact charged particle therapy systems are described. An energy selection system (“ESS”) that can be positioned proximate the patient is provided and enables the use of a monoenergetic charged particle beam. Using a monoenergetic charged particle beam, the beam delivery gantry can be made more compact than conventional charged particle gantries. Various configurations of static or rotatable gantries can be used.

### BACKGROUND

Ion, or charged particle, therapy (e.g., protons, helium, and carbon) has evolved to a highly sophisticated treatment delivery where Bragg peaks are deposited in the tumor volume. The range of the Bragg deposition depends on the ion's kinetic energy and is controlled by several different methods. It is undesirable to have long treatment times because the patient is required to be immobilized to ensure high quality treatment and long treatment times reduce overall efficiency for this limited resource.

In current charged particle therapy systems, an ion beam is generated and accelerated (i.e., its energy is increased) in an ion accelerator (e.g., a cyclotron, linac, or synchrotron). If the energy of the accelerated ion beam is not the desired energy then it is modified by an energy selection system (“ESS”) at the ion accelerator and then transported using magnets to the patient.

Some of the current technical limitations for charged particle therapy to achieve a sufficiently high dose rate to the clinical tumor volume include the inability to quickly change energy levels (i.e., range), the inability to achieve a high ion beam current at every desired energy level, and the inability to access shallow ranges (e.g., 0-4 cm) without the use of a secondary device. Changing the energy of the ion beam at the ion accelerator requires the beam transport magnets to also be adjusted in order to account for the change in ion beam energy, which is both time and cost prohibitive. The magnet switching time limits the energy switching time and requires additional technology to allow the magnet to switch their field strength. In addition, changing the energy of the ion beam at the ion accelerator can produce unwanted radiation that must be attenuated for radiation safety purposes.

Active scanning techniques, such as pencil-beam scanning, for charged particle therapy provide additional challenges for efficient energy selection. One such barrier for adoption of scanned pencil beam particle therapy is the increased cost over conventional radiation therapy. One large factor in this cost is the expense and complexity needed to produce and transport various energies from the accelerator to the patient. Another large cost is the facility needed for a charged particle device. Such facilities require walls that are sufficiently thick to meet radiation safety standards. The facilities also require a much larger footprint than conventional radiation therapy facilities.

Although ion beam energy can be varied at the nozzle using, for example, a range modulator (“RM”) wheel, this solution is not practical for use with pencil-beam scanning. For example double scatter systems use an RM wheel to modulate the pencil beam only on the central axis. Scattering devices placed downstream from the RM wheel are then used to spread the spot over the full field size, which then has to be collimated down to the field size needed. Such a configuration is not practical for pencil-beam scanning over a large field size because the necessary RM wheel would be prohibitively large. This configuration would also result in undesirable levels of spreading in the spot size of the ion beam.

### SUMMARY OF THE DISCLOSURE

The present disclosure addresses the aforementioned drawbacks by providing a charged particle beam delivery assembly for use in a charged particle therapy system. The beam delivery assembly includes an annular gantry having a bore extending along a longitudinal axis, and a plurality of beam transport magnets coupled to the gantry. Each beam transport magnet extends within a plane oriented relative to the longitudinal axis such that a charged particle beam entering the annular gantry along a first beam trajectory at an entrance point is bent towards the longitudinal axis by one or more magnetic fields generated by the beam transport magnets, thereby exiting the annular gantry at an exit point along a second beam trajectory.

It is another aspect of the present disclosure to provide an energy selection system for use in a charged particle therapy system. The energy selection system includes an annular support having a central bore extending along a longitudinal axis, and a plurality of chambers coupled to the annular support and azimuthally distributed about the longitudinal axis of the annular support. Each of the plurality of chambers has a volume extending along a radial direction outward from an outer surface of the annular support. The plurality of chambers are fluidically coupled so as to define a closed fluid dynamic system that is filled with a liquid absorber.

It is still another aspect of the present disclosure to provide an energy selection system for use in a charged particle therapy system, which includes a module that may be rotated and/or repeated for increased angular coverage. The module includes a chamber sized and dimensioned to span a plurality of different azimuthal angles, the chamber being filled with a liquid absorber. A corresponding plurality of pistons radially extend outward from the chamber and are configured to translate into and out of the chamber along a radial direction so as to decrease a thickness of the liquid absorber along the radial direction when moved into the chamber and increase the thickness of the liquid absorber when moved out of the chamber.

It is yet another aspect of the present disclosure to provide a scanning magnet assembly for use with a charged particle therapy system. The scanning magnet assembly includes a horizontal scanning magnet configured to steer an ion beam along a horizontal direction that is orthogonal to a longitudinal axis, and a vertical scanning magnet configured to steer an ion beam along a vertical direction that is orthogonal to the longitudinal axis. The vertical scanning magnet includes a first vertical scanning magnet part arranged on a first side of the horizontal magnet and a second vertical scanning magnet part arranged on a second side of the horizontal magnet opposite the first side along the longitudinal axis. The horizontal scanning magnet can be centered between the first vertical scanning magnet part and the second vertical scanning magnet part along the longitudinal axis such that the vertical scanning magnet and the horizontal scanning magnet have a common virtual source position.

It is another aspect of the present disclosure to provide a combined medical imaging and charged particle therapy system. The combined system includes a charged particle beam delivery assembly and a medical imaging system arranged proximate the charged particle beam delivery assembly along a longitudinal axis. The charged particle beam delivery assembly includes an annular gantry having a bore extending along a longitudinal axis, and a plurality of beam transport magnets coupled to the gantry. Each beam transport magnet extends within a plane oriented relative to the longitudinal axis such that a charged particle beam entering the annular gantry along a first beam trajectory at an entrance point is bent towards the longitudinal axis by one or more magnetic fields generated by the beam transport magnets, thereby exiting the annular gantry at an exit point along a second beam trajectory. The medical imaging system is arranged proximate the annular gantry along the longitudinal axis.

It is still another aspect of the present disclosure to provide a rotatable fluid coupling that includes a fixed face plate and a rotary face plate. The fixed face plate has an external side and an internal side, where a first plurality of concentric grooves is formed in the internal side of the fixed face plate. The rotary face plate also has an external side and an internal side, where a second plurality of concentric grooves is formed in the internal side of the rotary face plate. The fixed face plate and the rotary face plate are arranged relative to each other such that the first plurality of concentric grooves is interlocked with the second plurality of concentric grooves. The rotary face plate and the fixed face plate are coaxial with a rotation axis about which the rotary face plate is rotatable. A fluid flow path is defined between the internal side of the fixed face plate and the internal side of the rotary face plate between the first and second plurality of grooves such that when interlocked the first and second plurality of grooves reduce leakage of cryogen from the fluid flow path.

It is yet another aspect of the present disclosure to provide a charged particle therapy system that includes a rotatable annular gantry having a bore extending at least partially along a longitudinal axis; a plurality of beam transport magnets coupled to the gantry; an energy selection system coupled to the gantry; a rotatable electrical connection coaxial with the longitudinal axis and rotatably coupled to the annular gantry and in electrical communication with the plurality of beam transport magnets and the energy selection system; a scanning magnet assembly coaxial with the longitudinal axis and comprising at least one scanning magnet that generates a magnetic field that steers the charged particle beam in at least one direction thereby adjusting a position of the exit point of the charged particle beam as it exits the gantry; and a labyrinth cryogen face seal coaxial with the longitudinal axis and rotatably coupled to the rotatable electrical connection. Each beam transport magnet extends within a plane oriented relative to the longitudinal axis such that a charged particle beam entering the annular gantry along a first beam trajectory at an entrance point is bent towards the longitudinal axis by one or more magnetic fields generated by the beam transport magnets, thereby exiting the annular gantry at an exit point along a second beam trajectory. The energy selection system includes a chamber sized and dimensioned to span a plurality of different azimuthal angles, the chamber being coupled to the annular gantry and filled with a liquid absorber, and a corresponding plurality of pistons radially extending outward from the chamber. The pistons are configured to translate into and out of the chamber along a radial direction so as to decrease a thickness of the liquid absorber along the radial direction when moved into the chamber and increase the thickness of the liquid absorber when moved out of the chamber. The labyrinth cryogen face seal includes a fixed face plate opposing a rotary face plate that define therebetween a fluid flow path that provides continuous fluid coupling while the labyrinth cryogen face seal is rotated about the longitudinal axis.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1A shows an example charged particle therapy system, which can implement the downstream variable thickness energy selection systems described in the present disclosure.

FIG. 1B shows a view of the gantry of the charged particle therapy system of FIG. 1A.

FIG. 1C shows another example charged particle therapy system, which can implement a compact beam transport gantry such as those described in the present disclosure.

FIG. 1D shows another example charged particle therapy system, which can implement a compact beam transport gantry with a combined medical imaging system, as described in the present disclosure.

FIG. 2A shows an example of an azimuthal liquid energy selection system (“LESS”) design composed of a single module that spans two azimuthal angles.

FIG. 2B shows an example of an azimuthal LESS design composed of two modules that span four azimuthal angles.

FIG. 2C shows an example azimuthal LESS design spanning twelve azimuthal angles and fully encircling a central bore, in which the effective thickness of the energy selection system is varied by changing the volume of liquid absorber in one or more of a plurality of azimuthally distributed chambers of a closed fluid dynamic system.

FIG. 3 shows another example of a module that can be used in some configurations of an azimuthal LESS assembly, in which a single chamber is arranged to cover two different azimuthal beam trajectory angles.

FIG. 4 is a cross-sectional view of the azimuthal LESS module shown in FIG. 3.

FIG. 5 is a schematic illustration of an example fluidic coupling of two chambers used in some configurations of an azimuthal LESS assembly.

FIG. 6 shows an example vertex LESS assembly configured for selecting the energy of an axially directed charged particle beam.

FIGS. 7A and 7B show an example operation of the LESS assembly of FIG. 6.

FIG. 8A shows an example beam transport magnet gantry in accordance with some embodiments described in the present disclosure, in which the beam transport magnets fully encircle the bore of the gantry.

FIG. 8B shows an example beam transport magnet gantry in accordance with some other embodiments described in the present disclosure, in which the beam transport magnets partially encircle the bore of the gantry.

FIG. 9 shows an example of an azimuthal sector beam transport magnet gantry in accordance with some embodiments of the present disclosure.

FIG. 10 shows an example of a gradient scanning gantry in accordance with some embodiments of the present disclosure.

FIG. 11 shows an example of a radial sector beam transport magnet gantry in accordance with some embodiments of the present disclosure.

FIG. 12 shows an example of vertically scanning a charged particle beam to adjust its longitudinal exit point in a radial sector beam transport gantry.

FIGS. 13A-13D illustrate examples of a radial sector beam transport gantry together with an azimuthal LESS and vertex LESS assembly.

FIGS. 14A-14D illustrate examples of a compact charged particle therapy system utilizing a radial sector beam transport gantry having twelve radial sectors together with an azimuthal LESS and vertex LESS assembly.

FIGS. 15A and 15B illustrate a combined medical imaging and charged particle therapy system, utilizing an azimuthal sector beam transport and/or gradient scanning gantry as described in the present disclosure.

FIGS. 16A and 16B illustrate an example scanning magnet assembly that includes a split vertical scanning magnet centered on a horizontal scanning magnet along the longitudinal direction such that the vertical and horizontal scanning magnets have a common virtual source position.

FIGS. 17A-17D illustrate various configurations of full and partial beam transport magnet assemblies and azimuthal energy selection systems described in the present disclosure.

FIGS. 18A and 18B illustrate an example radial sector beam transport magnet assembly having three beam transport magnets, an azimuthal LESS assembly having a two-angle coverage, and a vertex LESS assembly.

FIGS. 19A-19C illustrate examples of beam transport magnets having a negative K-value (FIG. 19A), a positive K-value (FIG. 19B), and a zero K-value (FIG. 19C).

FIGS. 20A and 20B shows an example of a beam delivery device of a charged particle therapy system that includes a rotatable annular gantry supporting radial sector beam transport magnets with a K0 configuration and a combined azimuthal and vertex liquid energy selection system.

FIG. 21 shows an example of a combined azimuthal and vertex liquid energy selection system having a c-shaped chamber, or reservoir, of liquid absorber.

FIG. 22 shows an example of an x-ray imaging system that is integrated with a charged particle therapy system and arranged radially offset from the energy selection system of FIG. 21.

FIGS. 23A and 23B show examples of rotatable electrical connections using one or more slip rings that can provide power and electrical communication to components of a charged particle therapy system having a rotatable gantry.

FIGS. 24A-24F illustrate various views of an example rotatable fluid coupling configured as a labyrinth face seal for supplying a cryogen between a compressor and one or more cryocoolers used in a charged particle therapy system.

FIGS. 25A and 25B shows another example of a beam delivery device of a charged particle therapy system that includes a rotatable annular gantry supporting radial sector beam transport magnets and a combined azimuthal and vertex liquid energy selection system.

### DETAILED DESCRIPTION

Described here are systems and methods that enable compact charged particle therapy systems, such as proton therapy systems or other ion therapy systems (e.g., helium ion, carbon ion). In general, the systems and methods described in the present disclosure make use of an energy selection system (“ESS”) that can be positioned proximate the patient. It is an advantage of the systems described in the present disclosure that, because energy selection can be provided close to the patient, the construction of the charged particle therapy system can be made more compact and/or less expensive.

As one example, the beam line components can be made simpler since the charged particle therapy system can include an accelerator that generates a monoenergetic beam of the same energy in each use. As a result, the beam line components can be designed and constructed to account for only that single energy level, reducing their complexity and cost. Similarly, the beam transport magnet gantry can also be made simpler, smaller, and less expensive. As described below in more detail, these technical advantages can result in a charged particle therapy system with a smaller overall footprint, enabling wider clinical adoption of charged particle therapies. As another example, the building shielding can be designed to allow for beam transport at a single energy level, such that reduced complexity and/or expense in the building shielding design can be realized.

In some aspects, a variable thickness ESS is provided, where the variable thickness ESS is configured to be placed proximate to the patient receiving treatment. For instance, the variable thickness ESS is configured to be placed proximate to the patient and between the patient and the charged particle beam delivery components of the charged particle therapy system. In general, the ESS described in the present disclosure includes a variable thickness ESS assembly, which is controllable to quickly and accurately vary the thickness of one or more absorbers (e.g., liquid absorber) that are positioned within the ion beam path, thereby allowing for rapid control of the energy selection of the ion beam proximate to the patient. Advantageously, the ESS is located proximate the patient and is, therefore, downstream of the charged particle generating system, the beam transport system, and the lateral beam shaping devices (e.g., scanning magnets). Because energy selection is performed after all of the beam transport elements, the charged particle generating system (e.g., the synchrotron, cyclotron, or other accelerator) can be configured to produce a monoenergetic beam rather than a spectrum of various energies. This greatly simplifies the overall design of the charged particle generating system and beam transport system, including power supplies. Therefore, using the ESS described in the present disclosure allows for a significant reduction in the cost of equipment, as well as installation, commissioning, and maintenance costs while maintaining desired beam quality. For instance, beam transport can be performed with a monoenergetic beam, which allows for a less complex beam transport control system to be used. Less complex beam transport control systems are safer and more economical.

In some other aspects, a gantry for supporting charged particle beam transport magnets is provided. In general, the gantry designs described in the present disclosure can include a static gantry having a smaller form factor than the beam gantries used in existing charged particle therapy systems. Current charged particle therapy systems use gantries that have dimensions typically on the order of 30 feet in length (i.e., depth) and 15 feet or more in radius (e.g., for conventional proton therapy systems), or on the order of 75 feet in length (i.e., depth) and 20 feet in radius (e.g., for carbon ion therapy systems), or the like. These current gantries require dedicated vaults that are multistory structures sized to accommodate the larger gantries. On the other hand, the gantries described in the present disclosure have a much smaller form factor, similar to conventional medical imaging systems. For instance, the gantries described in the present disclosure can have dimensions on the order of 4-6 or 6-15 feet in length (i.e., depth) and 5-8 feet in radius.

In still other aspects, combined charged particle therapy and medical imaging systems are provided. In general, the compact form factor of the charged particle therapy systems described in the present disclosure enable their combination with medical imaging systems, such as x-ray computer tomography (“CT”), magnetic resonance imaging (“MRI”), and/or positron emission tomography (“PET”) imaging systems.

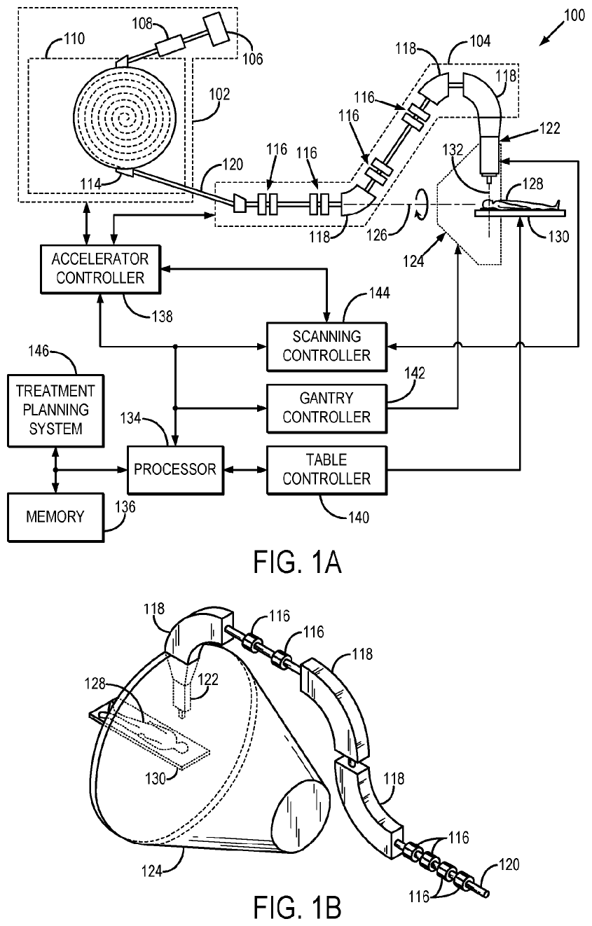

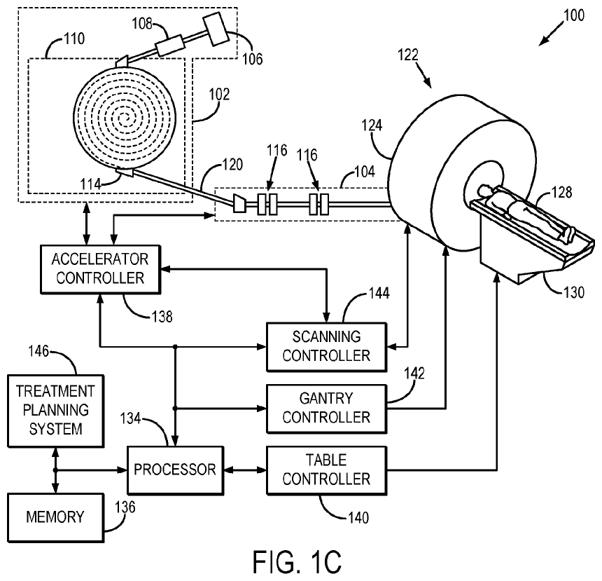

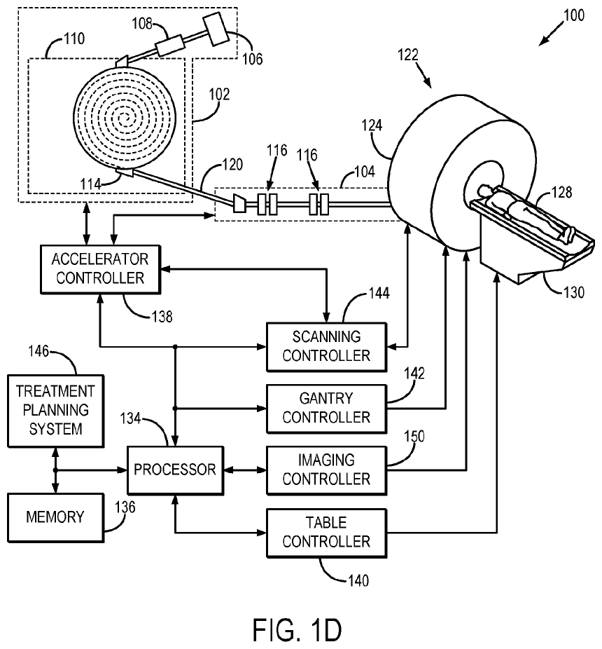

Referring now to FIGS. 1A-1D, an example of a charged particle therapy system 100, which may be a proton beam therapy system, an ion beam therapy system, or the like, is illustrated. Examples of charged particles for use with a charged particle therapy system include protons, ions, and/or molecules containing such particles. For example, charged particle therapy may include proton therapy and/or ion therapy (e.g., helium ion, carbon ion). An example charged particle therapy system 100 generally includes a charged particle generating system 102 and a beam transport system 104. By way of example, the charged particle generating system 102 may include a cyclotron; however, in other configurations the charged particle generating system 102 may include a synchrotron, a synchrocyclotron, or other suitable accelerator.

The charged particle generating system 102 includes an ion source 106, an injector 108, and an accelerator 110, such as a cyclotron. As a non-limiting example, when the accelerator 110 is a cyclotron, the injector 108 can include an axial injector, a radial injector, or another suitable external injection system suitable for use with a cyclotron. As another non-limiting example, when the accelerator 110 is a synchrotron, the injector 108 can be a linear accelerator (“linac”) or other suitable external injection system.

Ions generated in the ion source 106, such as hydrogen ions (i.e., protons), helium ions, or carbon ions, are accelerated by the injector 108 to form an ion beam that is injected into the accelerator 110. When the accelerator 110 is a cyclotron, the accelerator 110 can provide energy to the injected ion beam by way of a high frequency alternating voltage applied between two dees (e.g., D-shaped electrodes) inside a vacuum chamber. The dees are located between the poles of a large electromagnet that applies a static magnetic field perpendicular to the plane of the dees, which bends the ion beam into a circular trajectory within the vacuum chamber. When the alternating voltage is applied at the cyclotron resonance frequency of the particles in the ion beam, the ion beam is accelerated, which moves the ion beam path in an outward spiral trajectory while the particles in the ion beam are accelerated. When the accelerator 110 is a synchrotron, the accelerator 110 can provide energy to the injected ion beam by way of an acceleration cavity, where RF energy is applied to the ion beam. In the case of a synchrotron, quadrupole and dipole magnets are used to steer the ion beam about the accelerator 110 a number of times so that the ion beam repeatedly passes through the acceleration cavity.

After the energy of the ion beam traveling in the accelerator 110 has reached a preselected, desired energy level, which would typically be the optimized maximum energy (e.g., protons of 160 MeV, 250 MeV, or other suitable energy level), the ion beam is extracted from the accelerator 110 through an extraction deflector 114. Extraction may occur by way of bumping, or kicking, the ion beam to an outer trajectory so that it passes through a septum, or by way of resonance extraction.

The beam transport system 104 includes a plurality of focusing magnets 116 and steering magnets 118. Examples of focusing magnets 116 include quadrupole magnets, and examples of steering magnets 118 include dipole magnets. The focusing magnets 116 and steering magnets 118 are used to contain the ion beam in an evacuated beam transport tube 120 and to deliver the high energy ion beam to a beam delivery device 122 that is situated in a treatment room. In some other configurations, the beam transport system 104 can be simplified such that the ion beam can be delivered in a straight line from the charged particle generating system 102 to the beam delivery device 122, thereby simplifying the design of the focusing magnets 116 and steering magnets 118. For example, the beam transport system 104 may include only focusing magnets 116 in some implementations. In some configurations, the charged particle generating system 102 may be located within the same room as the beam delivery device 122 (i.e., the treatment room), or in a room adjacent to the treatment room.

The beam delivery device 122 is coupled or otherwise an integral part of a gantry 124, as will be described below in more detail. For instance, in some examples the beam delivery device 122 may be a nozzle of the ion therapy system. Alternatively, the beam delivery device 122 may be a portion of the gantry 124 that is configured to bend and deliver the ion beam to a treatment point or region within the patient 128. In these instances, scanning of the ion beam can be provided by scanning magnets located upstream of the gantry 124 (e.g., positioned between the charged particle generating system 102 and the gantry 124), as will be described in more detail below.

The gantry 124 may be a fixed gantry, or in some embodiments may be a rotatable gantry such that the beam delivery device 122 may be rotated about an axis of rotation 126 to deliver therapeutic radiation to a patient 128 positioned on a patient positioning device 130, which may be a patient table, a patient chair, or the like. When the gantry 124 is a fixed gantry (e.g., as shown in FIG. 1C), the ion beam can be steered and/or directed to deliver therapeutic radiation to the patient 128. For example, the gantry 124 can include one or more beam transport magnets that are configured to bend the ion beam from a first trajectory to a second trajectory. Examples of such gantry designs are described below in more detail.

When the gantry 124 is a rotatable gantry, the gantry 124 supports the beam delivery device 122 and deflection optics, including focusing magnets 116 and steering magnets 118, that form a part of the beam transport system 104. These deflection optics rotate about the rotation axis 126 along with the beam delivery device 122. Rotation of the rotatable gantry 124 may be provided, for example, by a motor. Alternatively, the beam delivery device 122 can be coupled to one or more fixed beams in one treatment room. In this case, the patient 128 can be immobilized on a patient positioning device, such as a robotic chair or table.

Alternatively, the beam delivery device 122 may be coupled to a non-rotatable support in a fixed beam configuration. In such a configuration, the position of the patient positioning device (e.g., patient table or chair) 130 can be adjusted to move the patient relative to the ion beam.

In some configurations, the accelerator 110 provides an ion beam to a plurality of beam delivery devices located in different treatment rooms. In such configurations, the beam transport system 104 may connect to a series of switchyards that may include an array of dipole bending magnets that deflect the ion beam to any one of a plurality of deflection optics that each leads to a respective beam delivery device in the respective treatment room.

The beam delivery device 122 is designed to deliver precise dose distributions to a target volume within a patient. In general, an example beam delivery device 122 includes components that may either modify or monitor specific properties of an ion beam in accordance with a treatment plan. For instance, the beam delivery device 122 can include one or more dose monitors (e.g., a main dose monitor and a backup dose monitor). In use, the dose monitor(s) can monitor the dose of the impinging ion beam, and can trigger interlocks that stop beam delivery when deviations from prescribed values are observed. These dose monitors and their associated control systems can be designed to measure very high beam currents from accelerators, such as cyclotrons, without loss of integrity.

The beam delivery device 122 may also, for example, include a device to spread or otherwise modify the ion beam position and profile, a dispersive element to modify the ion beam energy, and a plurality of beam sensors to monitor such properties. For example, scanning electromagnets may be used to scan the ion beam in orthogonal directions in a plane that is perpendicular to a beam axis 132. Advantageously, as described above the ESS described in the present disclosure are housed within the beam delivery device 122. Because the ESS is capable of selecting the desired energy of the ion beam, the range can be controlled and reduced without the need for a traditional range shifter (“RS”) within the beam delivery device 122. When the beam delivery device 122 is configured for pencil beam-scanning (“PBS”), additional monitors can also be included in the beam delivery device 122, such as beam profile and spot position monitors. In use, these monitors can trigger interlocks when the ion beam deviates from prescribed values.

In still other configurations, the beam delivery device 122 can include the gantry 124 and beam transport magnets supported by or housed within the gantry 124, as shown in FIG. 1C. In these instances, described in more detail below, the beam transport magnets are configured to bend the ion beam from a first trajectory to a second trajectory. In these instances, the gantry 124 may be a static or fixed gantry that supports an array of beam transport magnets. Alternatively, the gantry 124 may be a rotatable gantry that is configured to rotate about the longitudinal axis, and may support a full array of beam transport magnets, or one or more subsectors of beam transport magnets as described in more detail below. Components such as dose monitors may be integrated into the gantry 124, the energy selection system proximate the patient 128, or the like. Scanning of the ion beam is provided by scanning magnets, which may be located outside of the gantry 124 (e.g., between the charged particle generating system 102 and the gantry 124).

The charged particle therapy system 100 is controlled by a central controller that includes a processor 134 and a memory 136 in communication with the processor 134. An accelerator controller 138 is in communication with the processor 134 and is configured to control operational parameters of the charged particle generating system 102, including the accelerator 110 and the beam transport system 104. A table controller 140 is in communication with the processor 134 and is configured to control the position of the patient positioning device (e.g., patient table or chair) 130. A gantry controller 142 is also in communication with the processor 134 and is configured to control the rotation of the rotatable gantry 124 in those configurations where the gantry 124 can be rotated.

A scanning controller 144 is also in communication with the processor and is configured to control the beam delivery device 122. For instance, the scanning controller 144 can control the operation of beam transport magnets supported by or housed within the gantry 124. For example, as described below in more detail, the scanning controller 144 can adjust the magnetic field generated by the beam transport magnets such that the ion beam is steered to different treatment trajectories. The scanning controller 144 can also control the operation of one or more scanning magnets that are configured to adjust the entrance point of the ion beam into the gantry 124. Together, these scanning magnets and the beam transport magnets can control the final treatment trajectory of the ion beam as it exits the gantry 124.

The memory 136 may store a treatment plan prescribed by a treatment planning system 146 that is in communication with the processor 134 and the memory 136, in addition to control parameters or instructions to be delivered to the accelerator controller 138, the table controller 140, the gantry controller 142, and the scanning controller 144. The memory 136 may also store relevant patient information that may be utilized during a treatment session.

Before the ion beam is provided to the patient 128, the patient 128 is positioned so that the beam axis 132 intersects a treatment volume in accordance with a treatment plan prescribed by a treatment planning system 146. The patient 128 is positioned by way of moving the patient positioning device (e.g. patient table or chair) 130 into the appropriate position. The patient positioning device (e.g., patient table or chair) 130 position is controlled by the table controller 140, which receives instructions from the processor 134 to control the position of the patient positioning device (e.g., patient table or chair) 130. The rotatable gantry 124 is then set to a position dictated by the treatment plan so that the ion beam will be provided to the appropriate treatment location in the patient 128. The rotatable gantry 124 is controlled by the gantry controller 142, which receives instructions from the processor 134 to rotate the rotatable gantry 124 to the appropriate position. As indicated above, the position of the ion beam within a plane perpendicular to the beam axis 132 may be changed by the beam delivery device 122. The beam delivery device 122 is instructed to change this scan position of the ion beam by the scanning controller 144, which receives instruction from the processor 134. For example, the scanning controller 144 may control scanning electromagnets located in the beam delivery device 122 to change the scan position of the ion beam.

As described below in more detail, in some embodiments a medical imaging system can be integrated or otherwise combined with the beam delivery device 122. For example, a magnetic resonance imaging (“MRI”) system, an x-ray imaging system (e.g., an x-ray computed tomography (“CT”) system, a portal imaging system, etc.), a positron emission tomography (“PET”) imaging system, or the like, can be integrated or otherwise combined with the beam delivery device 122. In these instances, the particle therapy system 100 can also include an imaging controller 150 that is configured to control the operation of the integrated medical imaging system, as shown in FIG. 1D. Examples of imaging controller 150 hardware components and their operation are described below in more detail.

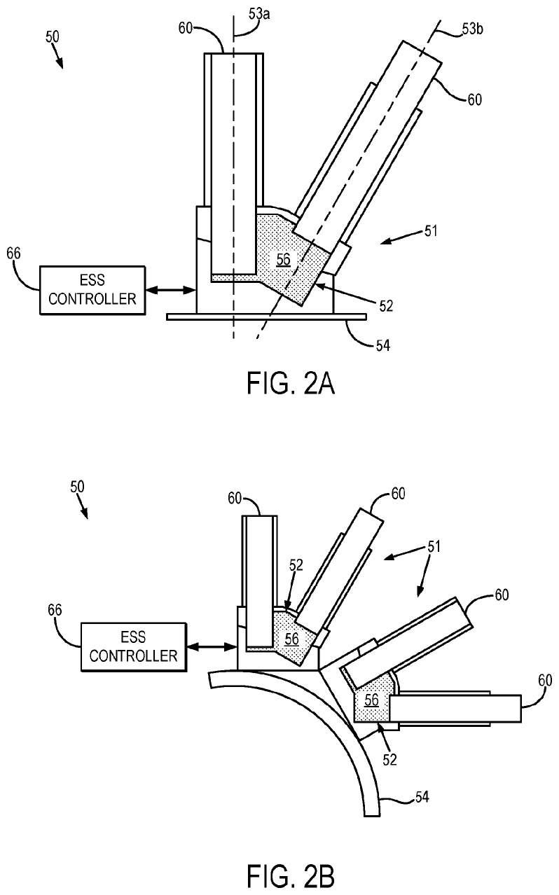

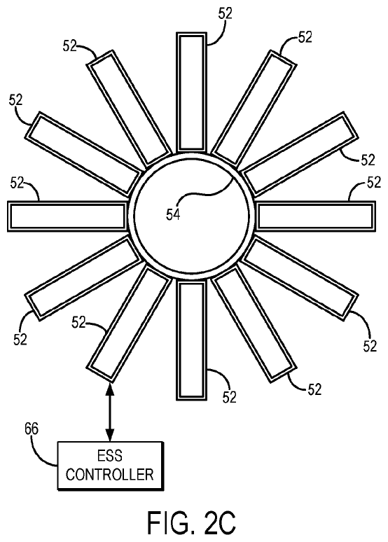

Referring now to FIGS. 2-6, example energy selection systems 50 according to some embodiments of the present disclosure are shown. In these embodiments, the energy selection system 50 implements a liquid energy selection system (“LESS”) design, in which the thickness of a liquid (or other fluid) absorber positioned within the beam path of the ion beam is varied under control of an energy selection system (“ESS”) controller 66.

The energy selection system 50 is constructed as a closed fluid dynamic system. In general, the energy selection system 50 includes a plurality of chambers 52 each filled with a volume of liquid absorber 56. The volume of each chamber 52 can be adjusted to vary the thickness of liquid absorber 56 along a beam path 53 through the respective chamber 52. In some embodiments, adjusting the volume of liquid absorber 56 in one chamber 52 will also adjust the volume of liquid absorber in an adjacent, fluidically coupled chamber 52. For instance, reducing the volume of liquid absorber 56 in one chamber 52 will cause the volume of liquid absorber 56 in one or more adjacent chambers 52 to increase. In other embodiments, a chamber 52 can be constructed to span more than one azimuthal angle for ion beam treatment and the thickness of liquid absorber 56 along a particular ion beam path 53 through the chamber 52 (e.g., corresponding to one azimuthal angle) can be varied by decreasing the volume of liquid absorber 56 along the ion beam path 53 (e.g., beam path 53a) while increasing the thickness of the liquid absorber 56 along an adjacent ion beam path 53 (e.g., beam path 53b). In still other embodiments, an external reservoir can be provided such that when the volume of liquid absorber is reduced in a chamber 52 the excess liquid absorber is returned to the reservoir for temporary storage.

An energy selection system 50 having an azimuthally distributed configuration is shown in FIGS. 2-5. In some configurations, the energy selection system 50 is constructed as a module 51 that spans an azimuthal range smaller than a full 360 degree of coverage. As shown in FIG. 2A. In these instances, the module 51 can be rotated about the longitudinal axis to cover additional azimuthal angles. The module 51 can be rotated about an annular support 54, which in some instances may be mounted within a gantry housing or the like. As shown in FIG. 2B, a module 51 can be repeated to span a larger azimuthal range with the energy selection system 50, and in some instances may be repeated to form a full array, as shown in FIG. 2C.

Each module 51 generally includes a chamber 52 that is filled with a liquid absorber 56 that includes liquid or other fluid that has appropriate radiation properties that ensures consistent energy absorption per unit density, has thermal characteristics that maintain quality under operational conditions, and has fluid dynamic properties that allow precise control of the variable thickness under high speed changes. For example, the liquid absorber 56 can be a high-density liquid with optimal viscosity and vapor pressure, and low reactivity properties. As one non-limiting example, the liquid absorber 56 can include a solution containing lithium heteropolytungstate (“LST”) or a solution containing a high proportion of hydrogen, such as glycerol and related liquids. The liquid absorber 56 may also be water. Alternatively, the liquid absorber 56 can include liquid metals, such as mercury or gallium-based alloys.

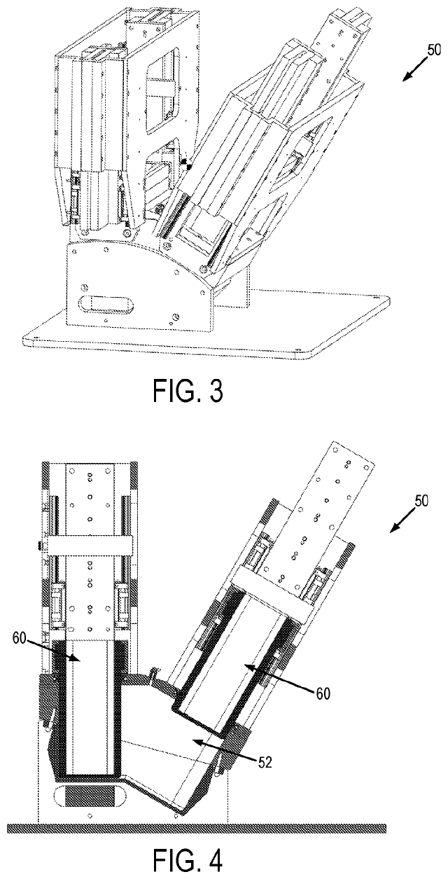

FIGS. 3 and 4 illustrate example configurations for a module 51 that can be used in an azimuthal energy selection system 50, such as those schematically shown in FIGS. 2A-2C. FIG. 4 shows a cross-section through the module 51 illustrated in FIG. 3. In the embodiment illustrated in FIGS. 3 and 4, the energy selection system 50 is constructed such that each chamber 52 spans two different azimuthal angles corresponding to two different potential ion beam treatment trajectories. A pair of pistons 60 move into and out of the chamber 52 to adjust the thickness of the liquid absorber 56 present along the different potential ion beam trajectories. By way of example, a linear actuator or motor can be used to translate the pistons under control of the ESS controller 66. As described above, a plurality of such modules 51 can be arranged about the circumference of the annular support 54 to form a partial or full array of modules 51. In these instances, each module 51 is its own closed fluid dynamic system, such that the chambers 52 of adjacent modules 51 are not fluidically coupled to each other.

In the illustrated embodiment of FIG. 2C, the energy selection system 50 includes twelve modules 51 or chambers 52 that are azimuthally distributed about the circumference of an annular support 54. More generally, the energy selection system 50 will include a plurality of chambers 52 that are azimuthally distributed about the circumference of the annular support 54, which may include fewer than twelve or more than twelve chambers 52. The total number of chambers 52 can be designed to match the number of azimuthal angles to which an ion beam can be steered by the beam delivery system 122 and/or gantry 124 of the charged particle therapy system 100. Alternatively, as described above one or more modules 51 can be configured to rotate about the longitudinal axis, such that the module(s) 51 can be rotated through the number of azimuthal angles to which an ion beam may be steered by the beam delivery system 122 and/or gantry 124 of the charged particle therapy system 100.

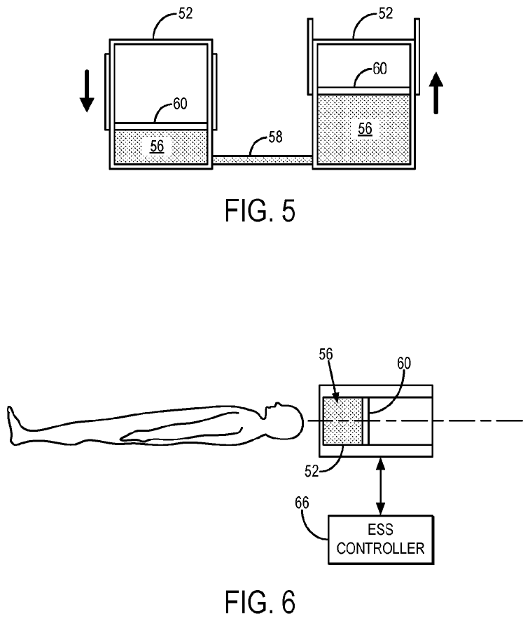

As schematically shown in FIG. 5, in some embodiments each chamber 52 of the energy selection system 50 may span only a single azimuthal angle. As described above, the liquid absorber 56 can be moved into and out of the chambers 52, such as by mechanical retraction and expansion or other fluidic control. The change in thickness can be accomplished by allowing the thickness of the liquid absorber 56 in a given chamber 52 of the energy selection system 50 to mechanically change. As also described above, in some embodiments, increasing the volume of liquid absorber 56 in one chamber 52 will cause a decrease in the volume of liquid absorber 56 in one or more adjacent chambers 52. In this way, an external reservoir is not needed for the energy selection system 50. Rather, the chambers 52 not in the ion beam path 53 can hold the excess volume of liquid absorber in the closed fluid dynamic system of the energy selection system 50.

As schematically shown in FIG. 5, in some embodiments adjacent chambers 52 can be fluidically coupled by a fluid coupling 58. A piston 60 may be translated along the longitudinal axis of a first chamber 52 to reduce the volume of liquid absorber 56 in that chamber 52. The excess liquid absorber 56 is then caused to flow into an adjacent chamber 52 where it acts upon the piston 60 in that chamber 52 to expand the volume within the chamber 52 to accommodate the excess liquid absorber 56. As described above, the piston 60 may be translated using a linear actuator or other motor operating under the control of the ESS controller 66.

Thus, in general, each chamber 52 is an expandable enclosure whose shape can change as the volume of the liquid absorber 56 within the chamber 52 is changed, or whose shape can be changed such that the volume of liquid absorber 56 within the chamber 52 adjusts to accommodate the different shape and/or volume of the chamber 52. As one non-limiting example, the chamber 52 can be shaped as a generally rectangular prism-shaped volume. In other instances, the chambers 52 can have cylindrical shapes, or other suitable shapes. For instance, as shown in FIGS. 3 and 4, the chambers 52 can have an arbitrary shape that accommodates spanning more than one azimuthal angle.

In use, the mechanical expansion and retraction of the volume of the chambers 52 can be controlled under instructions from the ESS controller 66 to adjust the thickness of a selected one of the chambers 52 by changing the volume of the liquid absorber 56 in the selected chamber 52. Because the energy selection system 50 is constructed as a closed system, changing the volume of liquid absorber 56 in one of the chambers 52 by moving liquid absorber 56 into the selected chamber 52 from one or more adjacent chambers 52 will increase the amount of liquid absorber 56 in the selected chamber 52. In this way, the amount of liquid absorber 56 through which the ion beam passes can be varied very rapidly, thereby allowing for an energy selection of the ion beam by controlling the thickness of one or more selected chambers 52. The change in energy selection (i.e., range selection) can be very small, such that the change can practically be a continuous change in range while the ion beam is continuously on.

The energy selection system 50 can be controlled by the ESS controller 66 that is operable in response to instructions received from the processor 134 to control the operation of the energy selection system 50. For instance, the ESS controller 66 can operate the fluidic systems that move the liquid absorber 56 into and out of the chambers 52. In use, liquid absorber 56 is moved into and out of the chambers 52 in order to adjust the thickness of liquid absorber 56 positioned within the beam path 53, thereby adjusting the energy of the ion beam to a desired energy level at the exit of the energy selection system 50. The effective thickness of the energy selection system 50 can be varied under control of the ESS controller 66 according to a prescribed radiation treatment plan.

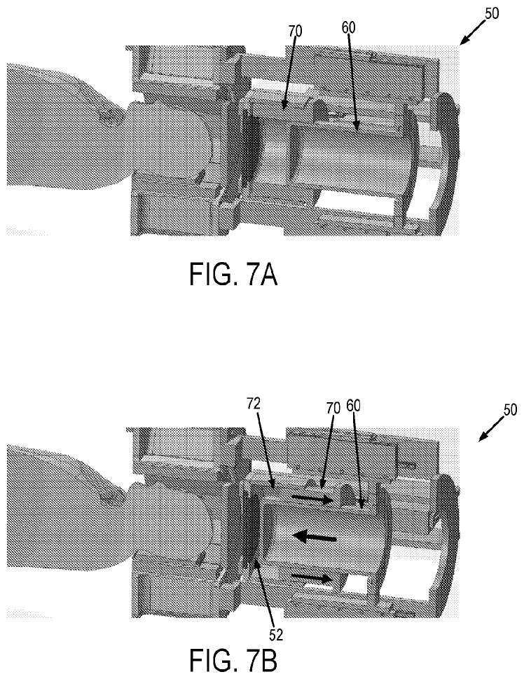

FIGS. 6, 7A, and 7B illustrate an example of an energy selection system 50 in a vertex configuration. In this example, the energy selection system 50 includes a single chamber 52 filled with a liquid absorber 56. Adjusting the volume of liquid absorber 56 within the chamber 52 will adjust the thickness of liquid absorber 56 within the ion beam path. In this configuration, the energy selection system 50 is configured to be positioned proximate the patient along the axial (i.e., longitudinal) direction. Translating the piston 60 increases or decreases the volume within the chamber 52, which then causes an increase or decrease, respectively, in the liquid absorber 56 within the chamber 52. When the piston 60 is translated to decrease the volume within the chamber 52 the excess liquid absorber 56 can be moved into an external reservoir, or to a different portion (e.g., an additional chamber) of the energy selection system 50 that is outside of the ion beam path. The piston 60 can be translated using a linear actuator or other motor operating under the control of the ESS control 66.

For example, as shown in FIGS. 7A and 7B, the energy selection system 50 can include a chamber 52 that is filled with liquid absorber 56. An annular collar 70 is arranged about the circumference of the piston 60, such that the piston 60 and annular collar 70 are able to translate past each other along the longitudinal axis. When the piston 60 is translated axially towards the patient the liquid absorber 56 in the path of the piston 60 is pushed laterally (e.g., radially) outward where it acts upon the annular collar 70 to push the annular collar 70 in the opposite direction along the longitudinal axis as the piston 60. The liquid absorber 56 thus flows into the additional volume 72 as the annular collar 70 is pushed out of the way. As such, the excess liquid absorber 56 is moved out of the ion beam path while still maintaining a single chamber 52 containing the liquid absorber 56 in a closed fluid dynamic system.

Like the azimuthal energy selection system 50, the energy selection system 50 in the vertex configuration is controllable by an ESS controller 66. In configurations where both an azimuthal and vertex energy selection system 50 are used in a single charged particle therapy system 100, the same ESS controller 66 can be used to control the operation of both the azimuthal and vertex energy selection systems 50. Alternatively, separate controllers could be used to independently control the different energy selection systems 50.

As described above, in some embodiments the gantry 124 of the charged particle therapy system 100 can be configured as a fixed and/or rotatable beam transport magnet gantry that supports or otherwise houses magnets for bending an ion beam from a first ion beam trajectory at an entrance point to a second ion beam trajectory at an exit point. Advantageously, the gantry designs described in the present disclosure can include a fixed and/or rotatable gantry having a smaller form factor than the gantries used in existing charged particle therapy systems.

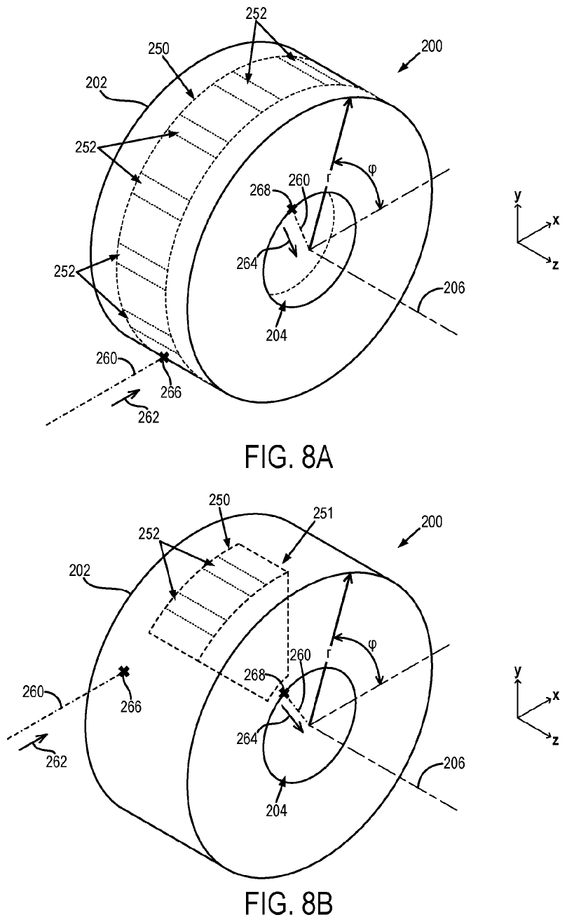

Referring now to FIGS. 8A and 8B, an example beam transport magnet gantry 200 for use in a charged particle therapy system 100 is shown. The gantry 200 generally includes an annular gantry housing 202 having a bore 204 extending along a longitudinal axis 206. In the illustrated embodiment, the longitudinal axis 206 is oriented along the z-direction, such that the x-y plane is an axial plane orthogonal to the longitudinal axis 206. A radial direction, r, is oriented along the radius of a circle within the axial plane, and an azimuthal direction, q, is the angular direction of rotation about the longitudinal axis 206 within the axial plane.

The gantry housing 202 houses, supports, or otherwise contains a beam transport magnet assembly 250. In general, the beam transport magnet assembly 250 contains a plurality of beam transport magnets 252 that are distributed within the gantry housing 202. For instance, the beam transport magnets 252 may be azimuthally distributed about the longitudinal axis 206. As will be described below in more detail, in some arrangements, the beam transport magnets 252 may be orientated in radial planes that are azimuthally distributed about the longitudinal axis 206. In some other arrangements, the beam transport magnets 252 may be oriented within azimuthally distributed axial sectors (i.e., sectors coplanar with one or more axial planes). Other configurations and arrangements of the beam transport magnets 252 are also possible, beam transport magnets 252 that are oriented at arbitrary or oblique angles with respect to the longitudinal axis 206, radial plane(s), axial plane(s), or the like. Combinations of different beam transport magnet orientations and geometries can also be implemented.

As shown in FIG. 8A, in some embodiments the beam transport magnets 252 extend fully around the circumference of the gantry housing 202. Alternatively, the beam transport magnets 252 may span a smaller angular range, as shown in FIG. 8B. In these instances, the beam transport assembly 250 is configured as a module 251 or subsector that can be rotated about the longitudinal axis 206 to move the beam transport magnets 252 into different azimuthal positions about the patient.

In general, the beam transport magnets 252 are configured to generate one or more magnetic fields and/or magnetic field gradients for bending an ion beam 260 from a first ion beam trajectory 262 to a second ion beam trajectory 264. The ion beam 260 enters the gantry housing 202 at an entrance point 266 along the first beam trajectory 262. The first ion beam trajectory 262 may be an initial ion beam trajectory (e.g., the trajectory of an ion beam exiting an accelerator or other charged particle generator, the trajectory of an ion beam after being scanned by one or more scanning magnets). The beam transport magnets 252 then bend or otherwise steer the trajectory of the ion beam 260 to the second ion beam trajectory 264 along which the ion beam 260 exits the gantry housing 202 at an exit point 268. The second ion beam trajectory 264 may be referred to as a treatment beam trajectory (e.g., the trajectory of the ion beam being directed towards a treatment point or region in a patient).

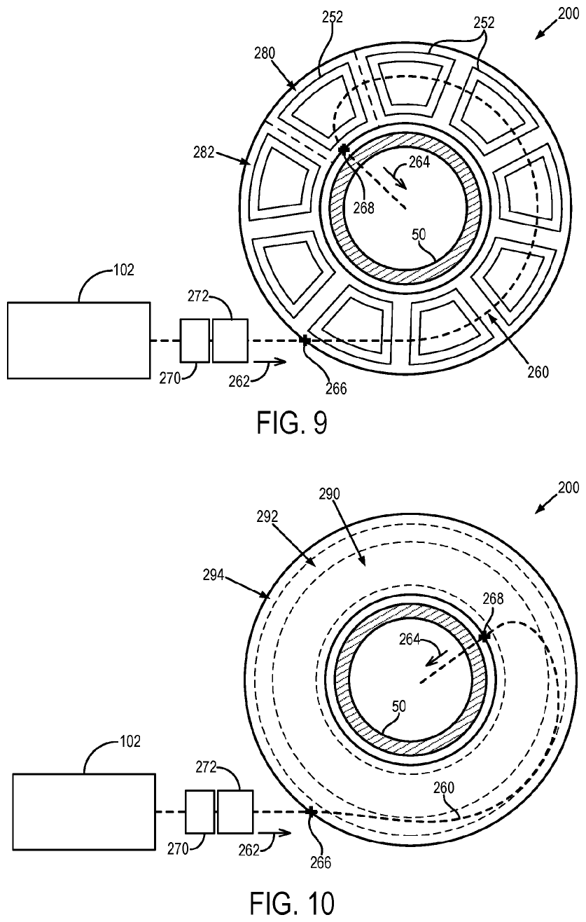

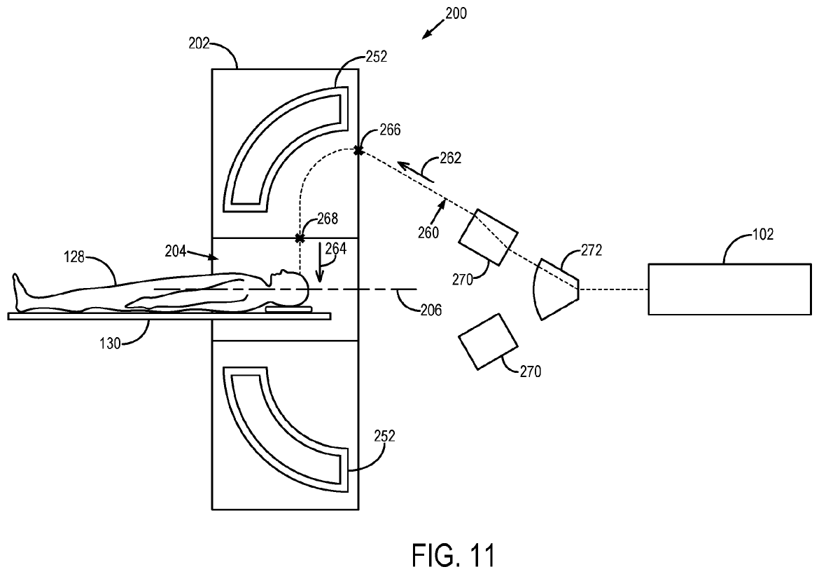

In some configurations, the first ion beam trajectory 262 can be oriented along a radial direction orthogonal to the longitudinal axis 206 (as shown in FIGS. 9 and 10). In these instances, the first ion beam trajectory 262 may be referred to as a radial trajectory. In some other configurations, the first ion beam trajectory 262 can be oriented along the longitudinal axis 206 (as shown in FIG. 11). In these instances, the first ion beam trajectory 262 may be referred to as an axial trajectory. In still other configurations, the first ion beam trajectory 262 can be oriented along an arbitrary direction relative to the longitudinal axis 206

In general, the second ion beam trajectory 264 is oriented along the radial direction or otherwise oriented inwardly toward the longitudinal axis 206, the inside of the bore 204, or the like. When using a proximate energy selection system 50, such as those described in the present disclosure, the second ion beam trajectory 264 can be directed and oriented such that the ion beam 260 passes through the energy selection system 50 before impinging upon the treatment point and/or region within the patient.

As shown in FIGS. 9-11, in some embodiments one or more scanning magnets 270, 272 external to the gantry 200 can also be utilized to steer the ion beam 260 from the first ion beam trajectory 262 to the second ion beam trajectory 264. In general, the one or more scanning magnets 270, 272 generate magnetic fields that can be controlled to steer the ion beam 260 onto a desired entrance point 266 such that the ion beam 260 will be bent by the beam transport magnets 252 within the gantry 200 to the desired exit point 268. In this way, the one or more scanning magnets 270, 272 can be used to adjust the axial and/or azimuthal position of the exit point 268 (and thus the treatment beam trajectory). For example, a first scanning magnet 270 can scan the ion beam in the axial direction (e.g., the z-direction) and a second scanning magnet 272 can scan the ion beam in the azimuthal direction. Advantageously, an azimuthal scanning magnet (e.g., scanning magnet 272) can be designed as a combination of x-scanning and y-scanning magnets into an R/PHI scanning magnet. It will be appreciated by those skilled in the art that various combinations of the scanning magnets and gantry designs described in the present disclosure can be combined for different effect or based on the therapeutic needs of the clinical site.

As shown in FIGS. 9-11, the scanning magnets 270, 272 are shown outside the gantry 200, which allows for the scanning of the ion beam 260 to be very fast. The first scanning magnet 270 scans the ion beam 260 in the horizontal direction, which will be in superior-inferior direction on the patient (i.e., along the longitudinal axis 206 of the gantry 200). In this configuration, the first scanning magnet 270 may be referred to as an SCMZ scanning magnet since it scans the exit point 268 of the ion beam 260 in the z-direction. The second scanning magnet 272 scan the ion beam 260 in the vertical direction, which will be in the azimuthal direction on the patient. In this configurations, the second scanning magnet 272 may be referred to as an SCMTH scanning magnet since it scans the exit point 268 of the ion beam 260 in the azimuthal direction.

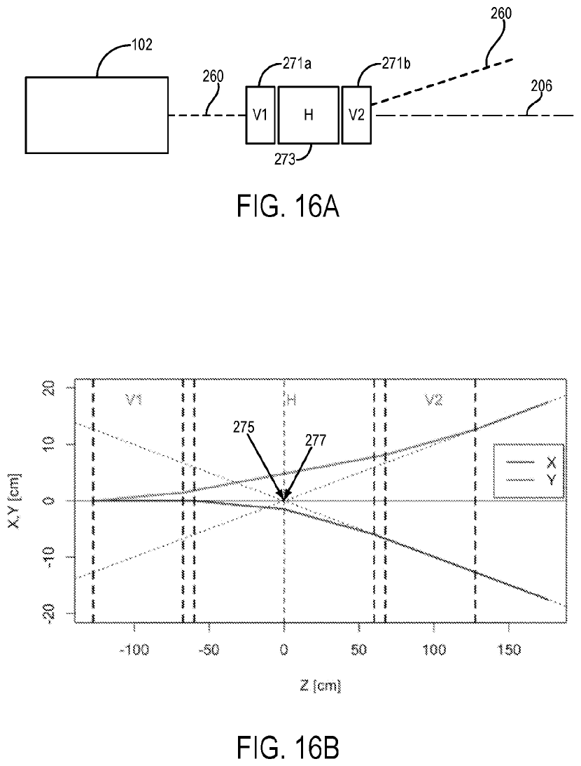

In some embodiments, the scanning magnets 270, 272 can be configured as an X-Y-X scanning magnet, as shown in FIGS. 16A and 16B. In this configuration, the scanning magnets are composed of a vertical scanning magnet 271 and a horizontal scanning magnet 273. The vertical scanning magnet 271 is composed of a first vertical scanning magnet part 271a and a second vertical scanning magnet part 271b arranged on opposite sides of the horizontal scanning magnet 273 along the longitudinal axis 206. The first and second vertical scanning magnet parts 271a, 271b are further arranged such that their virtual source position 275 is overlapped with the virtual source position 277 of the horizontal scanning magnet 273. For instance, the horizontal scanning magnet 273 can be centered between the first and second vertical scanning magnet parts 271a, 271b. In general, the first and second vertical scanning magnet parts 271a, 271b and the horizontal scanning magnet 273 are arranged along the longitudinal axis 206 such that as the ion beam 260 exits the charged particle generating system 102 the ion beam 260 first interacts with the magnetic field generated by the first vertical scanning magnet part 271a, then the horizontal scanning magnet 273, and then the second vertical scanning magnet part 271b. This scanning magnet configuration allows for simplification of the beam transport magnets 252 used to bend the ion beam 260 from the first ion beam trajectory 262 to the second ion beam trajectory 264.

When paired with an energy selection system 50 such as those shown in FIG. 2, different azimuthal angles of the treatment beam trajectory can be matched with different modules 51 and/or chambers 52 of the energy selection system, such that the energy selection system 50 is able to control the energy of the ion beam 260 before it reaches the patient along the selected treatment beam trajectory (i.e., the second ion beam trajectory 264). Additionally or alternatively, a vertex energy selection system 50 can also be provided to allow for energy selection of axially direction ion beams.

Referring to FIG. 9, an example gantry 200 is shown as having beam transport magnets 252 configured in azimuthally distributed sectors. The beam transport magnets 252 in the gantry 200 are oriented in the axial plane (e.g., orthogonal to the longitudinal axis 206) or in planes that are oblique to the longitudinal axis 206. The beam transport magnets 252 may be, for example, superconducting magnet coils.

In this configuration, the beam transport magnet(s) 252 in one sector 280 can be energized or otherwise configured to create a magnetic field having a different magnetic field strength than magnets 252 in the region 282 of the gantry 200 outside of the sector 280. This differential in magnetic field strength allows for the ion beam 260 to be bent towards the exit point 268 where the ion beam 260 will continue to propagate along the second ion beam trajectory 264. As a non-limiting example, the magnetic field within the sector 280 can be 5 T and the magnetic field in the region 282 outside of the sector 280 can be 2 T. By changing the location of the sector 280 with the stronger magnetic field, different azimuthal angles for the second ion beam trajectory 264 can be selected. The choice of sector 280 is thus determined by the desired gantry angle.

In this configuration, the ion beam 260 enters the gantry 200 at an entrance point 266 from a direction orthogonal to the longitudinal axis 206 of the gantry 200. The ion beam 260 can be steered onto different entrance points 266 by scanning the ion beam 260 using the first scanning magnet 270 (e.g., an SCMZ scanning magnet) and/or a second scanning magnet 272 (e.g., an SCMTH scanning magnet), as described above. For example, the scanning magnets 270, 272 deflect the initial beam trajectory to an entrance point 266 on the gantry 200 that will control the final treatment beam trajectory at the exit point 268 (e.g., by controlling the longitudinal exit point by scanning the beam horizontally, and controlling the azimuthal exit point by scanning the beam vertically).

Referring to FIG. 10, in some embodiments the beam transport magnets 252 can be configured to generate one or more magnetic field gradients within the housing 202 of the gantry 200. For example, in the illustrated embodiment the beam transport magnets 252 generate a magnetic field gradient from the inner radius of the housing 202 of the gantry 200 radially outward to the outer radius of the housing 202 of the gantry 200. The magnetic field gradient can be a continuous magnetic field gradient, or alternatively can be a discontinuous change in magnetic field from the central orbit to the outer and inner radii.

In the illustrated embodiment, the magnetic field gradient is composed of three regions of different magnetic field strength. As a non-limiting example, in the first region 290, the magnetic field strength can be at a first value, such as 10 T; the second region 292 can have a lower magnetic field strength, such as 2 T; and the third region 294 can have an intermediate magnetic field strength, such as 3 T. The ion beam 260 enters the stronger outer field (e.g., the region 290) by choosing an entrance point 266 and direction using the scanning magnets 270, 272. The choice of scanning magnet entrance angle and position is determined by the desired gantry angle.

Advantageously, in some embodiments, by establishing a magnetic field gradient within the housing 202 of the gantry 200, scanning magnets 270, 272 external to the gantry 200 may not be needed to define the exit point 268 of the ion beam 260. Instead, the magnetic field gradient(s) can be constructed such that the ion beam 260 is steered within the gantry 200 to the desired exit point 268.

Referring to FIG. 11, an example gantry 200 is shown as having beam transport magnets 252 arranged in different radial planes that are azimuthally distributed about the longitudinal axis 206 of the gantry 200. The beam transport magnets 252 may be, for example, superconducting magnet coils. A plurality of beam transport magnets 252 are provided, enabling a plurality of different azimuthal angles to be selected for the treatment beam trajectory. In some configurations, the radial sector gantry 200 can be referred to as having N different sectors, where N is the number of beam steering magnets 252 and, therefore, selectable azimuthal angles.

In this configuration, the beam transport magnet(s) 252 can be energized or otherwise configured to create a magnetic field that is oriented to bend the ion beam 260 from a generally axial trajectory to a radial trajectory directed inward toward the patient 128. The magnetic field can be a static magnetic field whose strength can be ramped up or down to provide treatment with different ion beam types. For example, the beam transport magnets 252 can have their magnetic field strength ramped to a first magnetic field strength when treating with a proton beam, a second magnetic field strength when treating with a helium ion beam, and a third magnetic field strength when treating with a carbon ion beam.

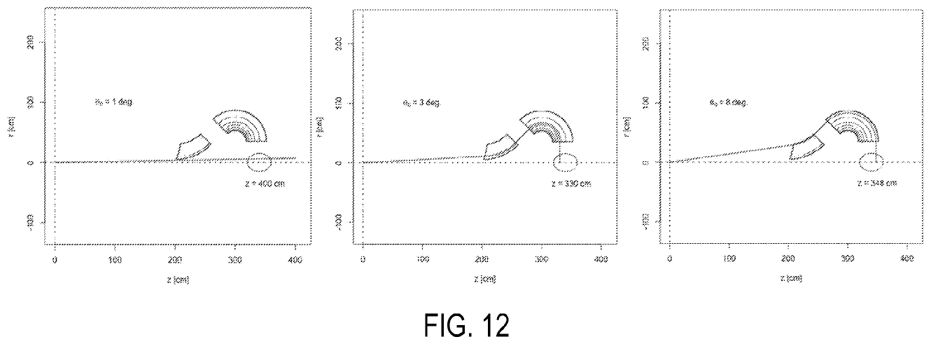

The ion beam 260 enters the gantry 200 at an entrance point 266 from a direction generally travelling along the longitudinal axis 206 or at one or more angles relative to the longitudinal axis 206. The ion beam 260 can be steered onto different entrance points 266 by scanning the ion beam 260 using the first scanning magnet 270 (e.g., an SCMZ scanning magnet) and/or a second scanning magnet 272 (e.g., an SCMTH scanning magnet), as described above, using the scanning magnet configuration shown in FIGS. 16A and 16B, or using other suitable scanning magnet configurations. For example, the scanning magnets 270, 272 (or scanning magnets 271, 273) deflect the initial beam trajectory to an entrance point 266 on the gantry 200 that will control the final treatment beam trajectory at the exit point 268 (e.g., by controlling the longitudinal exit point by scanning the beam vertically, and controlling the azimuthal exit point by scanning the beam and/or circumferentially). For example, FIG. 12 illustrates the effect of vertically scanning the initial ion beam trajectory as selecting a different axial position for the exit point 268. At low angles of deflection, the ion beam is axially directed toward the patient rather than bent by the beam transport magnets 252 into a radially directed trajectory.

The beam transport magnets 252 can additionally or alternatively be configured to create one or more magnetic field gradients, such that additional beam steering of the ion beam 260 can be provided by generating regions of different magnetic field strength within the housing 202 of the gantry 200 (e.g., as described above with respect to FIG. 10). As an example, the magnetic field gradient can be a radial magnetic field gradient, an axial magnetic field gradient, or a combination of both. Similarly, the magnetic field gradient can be a continuous magnetic field gradient or a discontinuous magnetic field gradient.

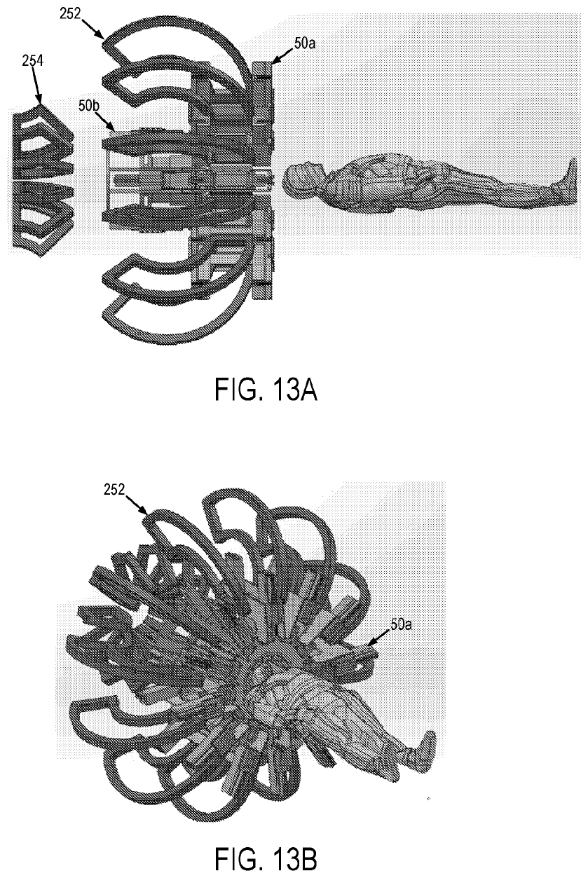

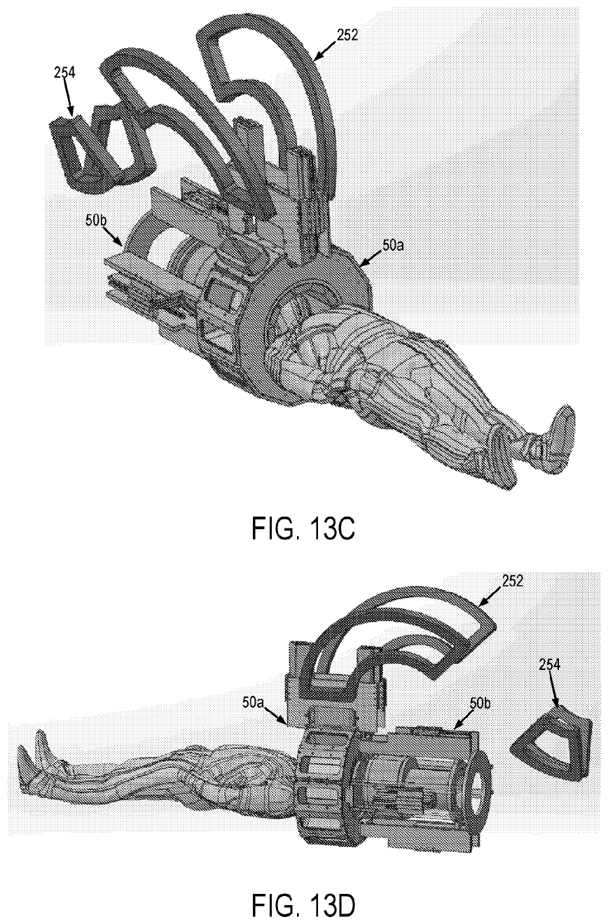

FIGS. 13A-13D show various views of an example radial sector beam transport gantry 200 in accordance with some embodiments of the present disclosure. In the illustrated embodiment shown in FIGS. 13A and 13B, the gantry 200 includes twelve azimuthally distributed beam transport magnets 252. As such, the gantry 200 may be used to deliver an ion beam 260 at twelve different azimuthal angles about the patient 128. In other embodiments, fewer than twelve azimuthally distributed beam transport magnets 252 can be used (e.g., those shown in FIGS. 13C and 13D). The beam transport magnets 252 are configured as focusing magnets providing an approximately 135 degree bend. A second set of matched beam transport magnets 254 is provided proximal to the first set of beam transport magnets 252 along the longitudinal axis 206 (i.e., the second set of beam transport magnets 254 is located closer to the charged particle generating system 102 than the first set of beam transport magnets 252). The second set of beam transport magnets 254 are configured as defocusing magnets that provide an approximately 45 degree bend.

In some configurations, the gantry 200 is a fixed gantry. In some other configurations, the gantry 200 is a rotatable gantry. For example, in the rotatable gantry configuration, the gantry 200 can be constructed to be rotated about the longitudinal axis 206 to provide additional control over the ion beam 260 delivery. The rotatable gantry can be rotated mechanically. For instance, the rotatable gantry can be rotated as a rate of 6 degrees rotation per second, or the like. As described above, the rotatable gantry can support an array of beam transport magnets 252 that fully encircle the patient 128 (as shown in FIGS. 13A and 13B), or can support a module 251 containing fewer beam transport magnets 252 that do not fully encircle the patient 128 (e.g., two or three beam transport magnets 252, as shown in 13C and 13D).

As shown in FIGS. 13A-13D, a first energy selection system 50a provides energy selection for radially oriented ion beam trajectories, and a second energy selection system 50b provides energy selection for axially oriented ion beam trajectories. For instance, the first energy selection system 50a can be an azimuthal energy selection system and the second energy selection system 50b can be a vertex energy selection system. Additionally or alternatively, other energy selections systems that can be arranged proximate the patient can also be used with the systems described in the present disclosure, such as those energy selection systems described in co-pending PCT Patent Application No. US2021/040358, which is herein incorporated by reference in its entirety.

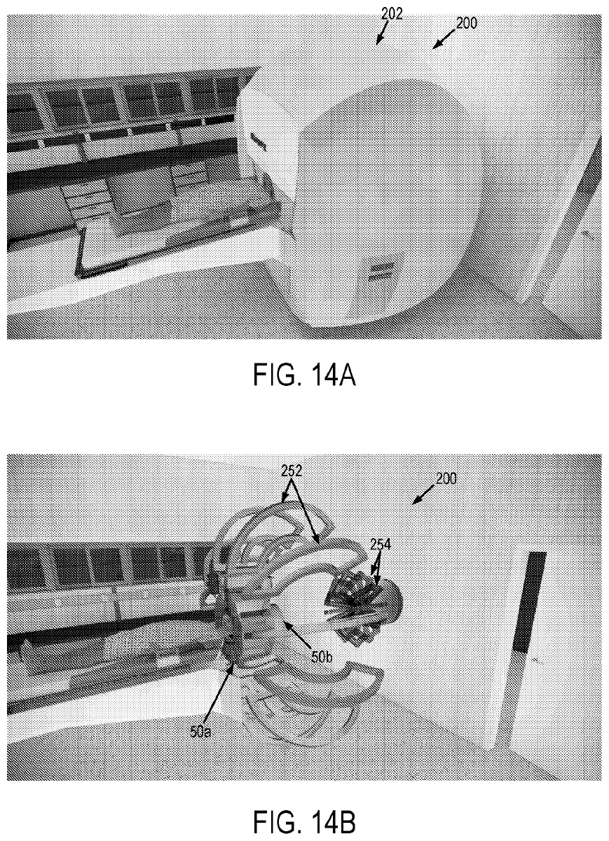

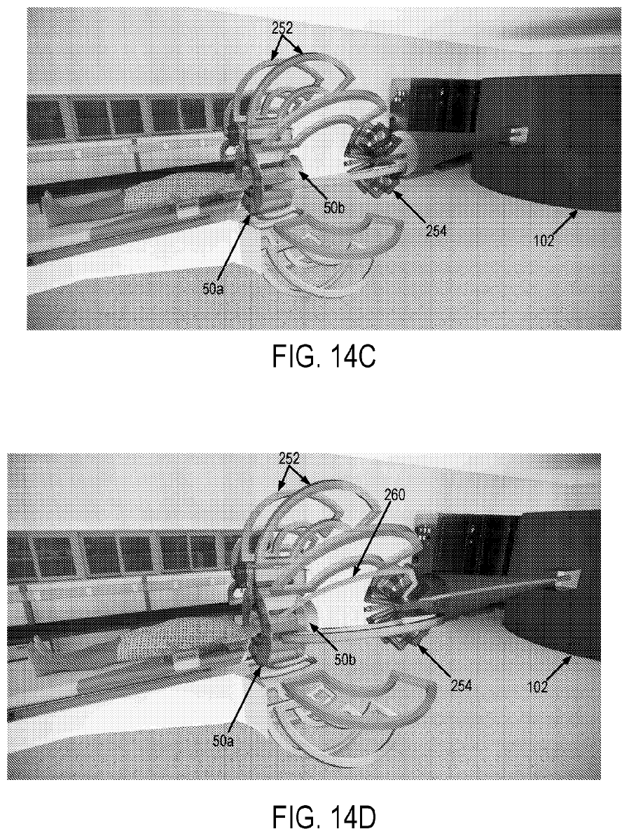

FIGS. 14A-14D show an example arrangement of a charged particle therapy system utilizing a radial sector gantry 200, such as those shown in FIGS. 13A-13D. FIG. 14A shows an example gantry 200 with its gantry housing 202 shown in a treatment room. FIG. 14B shows the same system with the gantry housing 202 removed for illustration purposes. In FIG. 14B, the energy selection systems 50a, 50b, first beam transport magnets 252, and second beam transport magnets 254 can all be seen. FIGS. 14C and 14D show the adjoining ion generation room housing the charged particle beam generating system 102, which may be a cyclotron, a synchrotron, or other accelerator. As shown, the compact design of the components described in the present disclosure enables the charged particle therapy system to be housed within a conventional clinical setting without the need for multistory gantry systems as in conventional charged particle therapy systems.

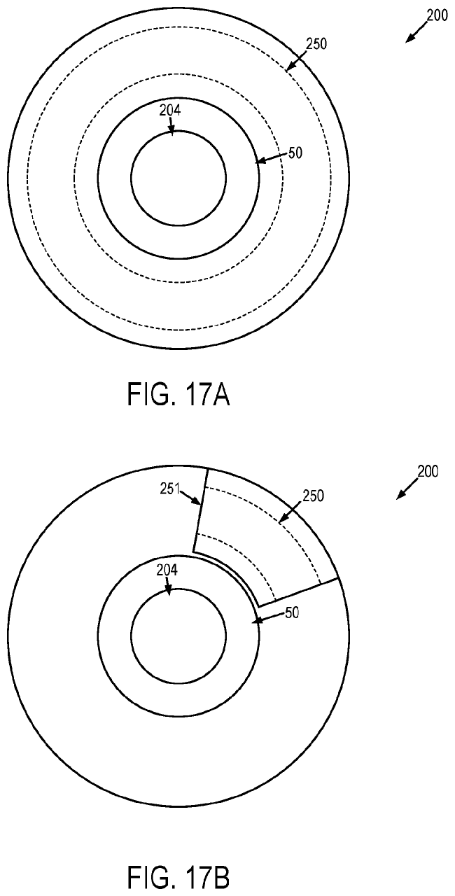

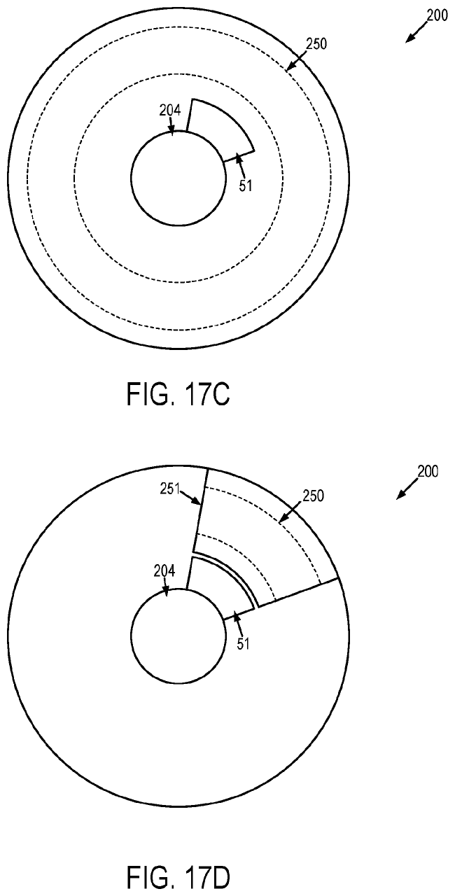

As described above, the charged particle therapy systems 100 described in the present disclosure can include a combination of different azimuthal LESS configurations for the energy selection system 50 and different gantry designs for the gantry 200 that supports the beam transport magnets 252. As shown in FIG. 17A, in one embodiment the gantry 200 can include beam transport magnets 252 that fully encircle the bore 204 of the gantry 200, together with an azimuthal LESS energy selection system 50 that also fully encircles the bore 204 of the gantry. The gantry 200 and the energy selection system 50 may be fixed, may be rotatable, or combinations thereof.

As shown in FIG. 17B, in some embodiments, the beam transport magnets 252 may not fully encircle the bore 204 of the gantry 200. In these instances, the beam transport assembly 250 is configured as one or more modules 251 that can be rotated about the longitudinal axis 206 of the gantry 200. The module(s) 251 may be supported within the gantry housing 202, as an example. In these instances, the azimuthal LESS energy selection system 50 may be fixed, or may be rotatable about the longitudinal axis 206 as well.

As shown in FIG. 17C, in some embodiments, the azimuthal LESS energy selection system 50 may not fully encircle the bore 204 of the gantry 200. In these instances, the energy selection system 50 is configured as one or more modules 51 that can be rotated about the longitudinal axis 206 of the gantry 200. The module(s) 51 may be supported within the gantry housing 202, as an example. In these instances, the beam transport assembly 250 and thus beam transport magnets 252 may be fixed, or may be rotatable about the longitudinal axis 206 as well.

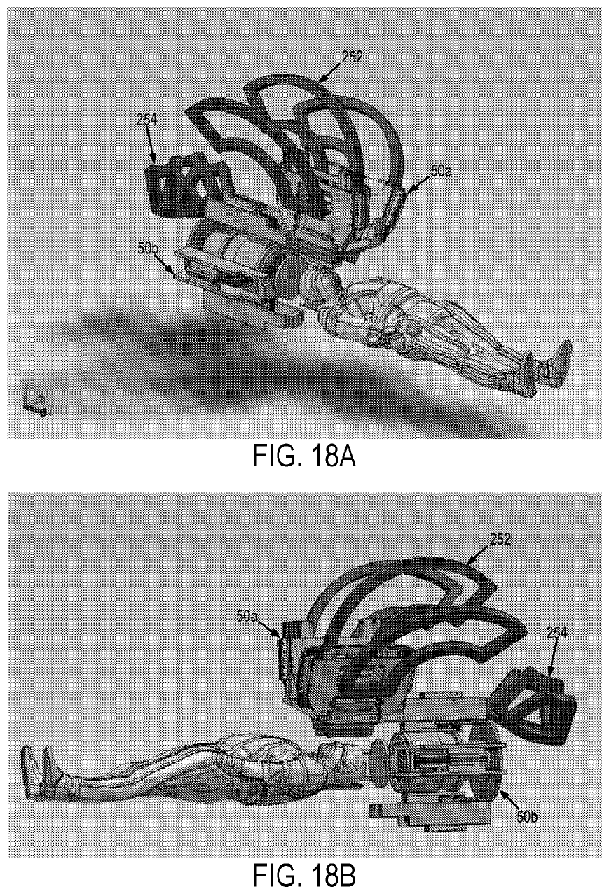

As shown in FIG. 17D, in some embodiments neither the beam transport magnets 252 nor the azimuthal LESS energy selection system 50 fully encircle the bore 204 of the gantry 200. In these instances, the beam transport assembly 250 is configured as one or more modules 251 that can be rotated about the longitudinal axis 206 of the gantry 200, and the energy selection system 50 is configured as one or more modules 51 that can be rotated about the longitudinal axis 206 of the gantry 200. The module(s) 251 of beam transport magnets 252 may be rotated independently of the module(s) 51 of the energy selection system 50, or may be rotated in unison. As a non-limiting example, the module 251 of the beam transport magnet assembly 250 can include beam transport magnets 252 arranged in a radial sector configuration. The module 251 may include two beam transport magnets 252, may include three beam transport magnets 252, or more. The module 51 of the azimuthal LESS energy selection system 50 can include a single chamber 52 spanning one azimuthal angle, two azimuthal angles, or more. An example configuration with three beam transport magnets 252 and a two-angle azimuthal LESS energy selection system 50a and a vertex LESS energy selection system 50b is illustrated in FIGS. 18A and 18B.

Advantageously, a compact particle accelerator and compact gantry can be combined into a single system, or a single accelerator can be used to serve multiple different gantries. The cost of a conventional charged particle therapy building is substantial due to the size and the shielding requirements of the equipment. Using the systems described in the present disclosure, the equipment is designed to be compact such that the equipment can fit on a single floor, as shown in FIGS. 14A-14D. In these configurations, the shielding can be reduced by manufacturing the shielding into the equipment itself.

The main sources of radiation beam losses for shielding requirements are from the accelerator, the transfer line using a fast Faraday cup and profile monitors, the energy selection system, and the patient. The radiation from the accelerator and the beam line components is generated from the ions incident on high-Z materials like iron. The radiation from the energy selection system and the patient is generated from the ions incident on low-Z materials like water, glycerol, tissue, and carbon. The secondary fast neutron spectrum from each of these has been studied and due to the design of the equipment the shielding requirements of the building can be minimized.

Typically, a charged particle therapy building is made of concrete and the concrete is made sufficiently thick to also serve as a radiation barrier. This is because of its structural use and moreover the cost effectiveness of using concrete as radiation shielding. Iron is commonly used for a return yoke and magnetic shielding; therefore, in situations where large amounts of iron are used the concrete shielding requirements for new installs can be tailored to the equipment. However, when installing into existing facilities, if the equipment is sufficiently compact then there is great benefit in the equipment being self-shielded over and above the iron. Both hydrogen and iron are good shields for neutrons, except that iron is transparent to neutrons in the region of 200 keV to 300 keV, so iron shielding should be followed by a layer of hydrogenous material in those instances.

There are several materials that can be used as shielding outside the iron magnetic shielding to provide this self-shielding, such as iron, steel, Ledite, polyethylene, borated polyethylene, and others.

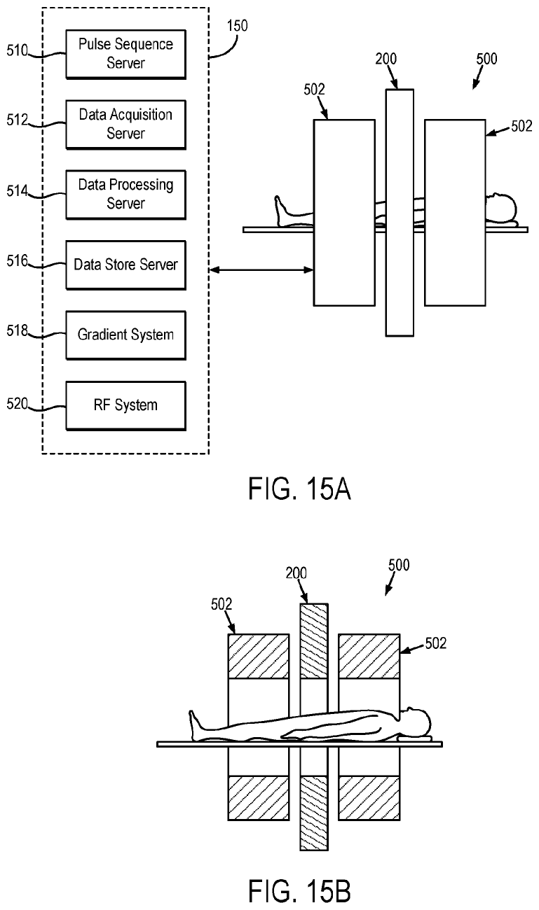

As shown in FIGS. 15A and 15B, another advantage of the compact charged particle therapy systems described in the present disclosure is that they can be integrated with medical imaging systems to provide a combined image guided charged particle therapy system 500. For instance, a gantry 200 (e.g., an azimuthal gantry such as the one illustrated in FIG. 9 or 10) can be arranged between two superconducting magnets 502 used to provide the polarizing main magnetic field in a split bore MRI system. Similarly, the gantry 200 could be integrated with other medical imaging systems, including x-ray CT imaging systems, which may include cone-beam CT (“CBCT”) systems; PET imaging systems; or the like.

Advantageously, such systems enable real-time feedback for charged particle treatments based on the anatomical and/or functional imaging that can be provided by the medical imaging system. For instance, because the medical imaging system and charged particle therapy system 500 are spatially coregistered, the medical imaging system can be used to acquire images of the patient without moving the patient from an alignment with the ion beam of the charged particle therapy system 500. In this way, images of the patient can be obtained while the patient is aligned with the ion beam, allowing for verification of correct beam positioning, dose monitoring, and the like.

In the illustrated example, the combined medical imaging system and charged particle therapy system 500 includes an MRI system. As described above, in such a configuration the operation of the MRI system can be controlled by an imaging controller 150 that includes various hardware electronics and/or components for operating the MRI system. For example, the imaging controller 150 may include an operator workstation coupled to different servers, including, for example, a pulse sequence server 510, a data acquisition server 512, a data processing server 514, and a data store server 516. The operator workstation and the servers 510, 512, 514, and 516 may be connected via a communication system, which may include wired or wireless network connections. The operator workstation may be implemented as a computer system including a display, one or more input devices (e.g., a keyboard, a mouse), and a processor. The processor may include a commercially available programmable machine running a commercially available operating system. The operator workstation provides an operator interface that facilitates entering scan parameters into the MRI system.

The pulse sequence server 510 functions in response to instructions provided by the operator workstation to operate a gradient system 518 and a radiofrequency (“RF”) system 520. Gradient waveforms for performing a prescribed scan are produced and applied to the gradient system 518, which then excites gradient coils in an assembly to produce the magnetic field gradients Gₓ, G_{y}, and G_{z} that are used for spatially encoding magnetic resonance signals. The gradient coil assembly forms part of a magnet assembly that includes a polarizing magnet(s) 502 and a whole-body RF coil.

RF waveforms are applied by the RF system 520 to the whole-body RF coil, or a separate local coil, to perform the prescribed magnetic resonance pulse sequence. Responsive magnetic resonance signals detected by the RF coil(s) are received by the RF system 520. The responsive magnetic resonance signals may be amplified, demodulated, filtered, and digitized under direction of commands produced by the pulse sequence server 510. The RF system 520 includes an RF transmitter for producing a wide variety of RF pulses used in MRI pulse sequences. The RF transmitter is responsive to the prescribed scan and direction from the pulse sequence server 510 to produce RF pulses of the desired frequency, phase, and pulse amplitude waveform. The generated RF pulses may be applied to the RF coil(s).

The RF system 520 also includes one or more RF receiver channels. An RF receiver channel includes an RF preamplifier that amplifies the magnetic resonance signal received by the RF coil(s) to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} ;$$

and the phase of the received magnetic resonance signal may also be determined according to the following relationship:

$$φ = {tan}^{- 1} ( \frac{Q}{I} ) .$$

The pulse sequence server 510 may receive control signals from the processor 134 or control system of the charged particle therapy system 100, which may be used by the pulse sequence server 510 to synchronize, or “gate,” the performance of the scan with the delivery of charged particle therapy to the patient.

The digitized magnetic resonance signal samples produced by the RF system 520 are received by the data acquisition server 512. In some scans, the data acquisition server 512 passes the acquired magnetic resonance data to the data processor server 514. In scans that require information derived from acquired magnetic resonance data to control the further performance of the scan or control of the charged particle therapy system 100, the data acquisition server 512 may be programmed to produce such information and convey it to the pulse sequence server 510. For example, during pre-scans, magnetic resonance data may be acquired and used to calibrate the pulse sequence performed by the pulse sequence server 510. As another example, navigator signals may be acquired and used to adjust the operating parameters of the RF system 520, the gradient system 518, or to control the delivery of charged particle therapy via the charged particle therapy system 100.

The data processing server 514 receives magnetic resonance data from the data acquisition server 512 and processes the magnetic resonance data in accordance with instructions provided by the operator workstation. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, or calculating patient motion (e.g., respiratory motion).

Images reconstructed by the data processing server 514 can be conveyed back to the operator workstation for storage. Real-time images may be stored in a data base memory cache, from which they may be output to a display for viewing by a user. The operator workstation may be used by an operator to archive the images or send the images via a network to other facilities.

The magnets in the beam transport assembly 250 (e.g., beam transport magnets 252, 254) can be constructed to have different effects on the charged particle beam, as will be described below in more detail. The magnetic field gradient normalized with respect to magnetic rigidity can be expressed as:

$$K = \frac{1}{{ρ}^{2}} + \frac{\partial {B}_{z} / \partial ρ}{( B ρ )} ; \qquad ( 1 )$$

where ρ is the radial distance; B_{z} is the magnetic field strength along the z-direction, which may be, for example, along the beam axis perpendicular to the radial direction; and “Bρ” is the magnetic rigidity, which is the effect of the magnetic fields on the motion of the charged particles in the charged particle beam. The beam transport magnets 252, 254 can be constructed to have different K-values to achieve different effects on the charged particle beam, such as to defocus the charged particle beam, to focus the charged particle beam, and/or to keep a parallel trajectory for the charged particle beam throughout the magnet. As one example, the geometry of the windings in the beam transport magnet can be designed to adjust the K-value of the magnet. As another example, the gap spacing between windings measured along the radial direction can be adjusted along the axial length of the beam transport magnet to adjust the K-value for the magnet. For instance, increasing the gap spacing can adjust the K-value towards negative values, whereas decreasing the gap spacing can adjust the K-value towards positive values.

A gantry 200 can be referred to as a KN gantry when the magnets in the beam transport assembly 250 (e.g., beam transport magnets 252, 254) have a negative K-value (i.e., K<0), such as where:

$$\frac{\partial {B}_{z}}{\partial ρ} < - \frac{( B ρ )}{{ρ}^{2}} . \qquad ( 2 )$$

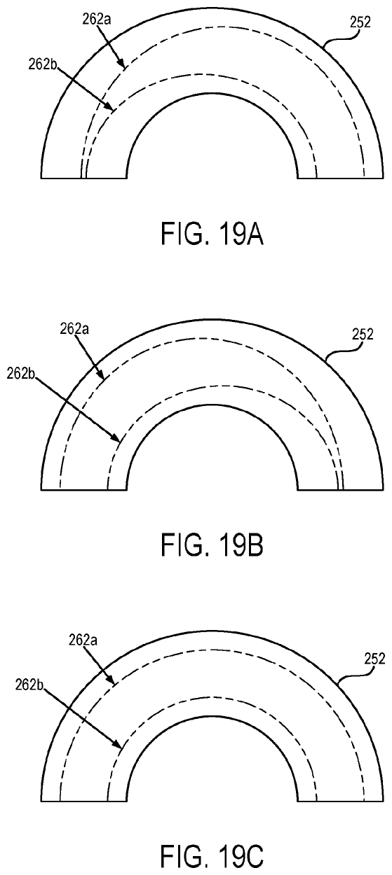

In these instances, the charged particle beam trajectories entering the beam transport magnet will be defocused. For instance, as shown in FIG. 19A, two beam paths 262a, 262b will diverge as the charged particle beams traverse the beam transport magnet(s) 252, 254. The KN gantry configuration has the advantage of increasing the field length for a given trajectory angle, even with a more compact beam transport assembly 250 (i.e., beam transport magnets with a shorter length). Advantageously, a KN gantry design can allow for a more compact gantry 200 since a little kick at the scanning magnet(s) 270, 272 can result in a large kick at the patient. This enables a larger scanning range without having to move the patient, and also with a smaller beam path. Additionally or alternatively, a simpler scanning magnet design can be used with the KN gantry configuration.

Similarly, the gantry 200 can be referred to as a KP gantry when the magnets in the beam transport assembly 250 (e.g., beam transport magnets 252, 254) have a positive K-value (i.e., K>0), such as where:

$$\frac{\partial {B}_{z}}{\partial ρ} > - \frac{( B ρ )}{{ρ}^{2}} . \qquad ( 3 )$$

In these instances, the charged particle beam trajectories entering the beam transport magnet will be focused. For instance, as shown in FIG. 19B, two beam paths 262a, 262b will converge as the charged particle beams traverse the beam transport magnet(s) 252, 254. Similar to the KN gantry, a KP gantry design can also allow for a more compact gantry 200, in addition to a simpler scanning magnet design.

When the magnets in the beam transport assembly 250 (e.g., beam transport magnets 252, 254) have a K-value equal to zero, the gantry can be referred to as a K0 gantry, such as where:

$$\frac{\partial {B}_{z}}{\partial ρ} = - \frac{( B ρ )}{{ρ}^{2}} . \qquad ( 4 )$$

In these instances, the charged particle beam trajectories entering the beam transport magnet will remain parallel to each other as the charged particle beams traverse the beam transport magnet(s), as shown by the two beam paths 262a, 262b in FIG. 19C. Similar to the KN and KP gantries, a K0 gantry design can also allow for a more compact gantry 200, in addition to a simpler scanning magnet design.

Although single K-value designs are descried above, in alternative examples the beam transport assembly 250 can be designed to include magnets having different K-values. For instance, a beam transport assembly 250 may include one or more beam transport magnets (e.g., beam transport magnets 252) with a different K-value than one or more other beam transport magnets (e.g., beam transport magnets 254). As an example, beam transport magnet(s) 252 may have a negative K-value (i.e., be configured as KN magnets) while beam transport magnet(s) 254 may have a zero or positive K-value (i.e., be configured as K0 or KP magnets). Various different combinations of KN, KP, and K0 magnets can be utilized in a particular beam transport assembly 250; that is, the beam transport assembly 250 does not need to be composed of only KN magnets, only KP magnets, or only K0 magnets, but combinations of different K-value magnets.

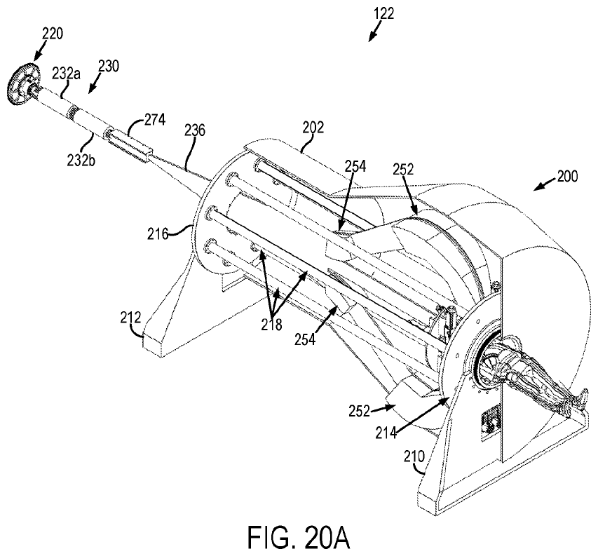

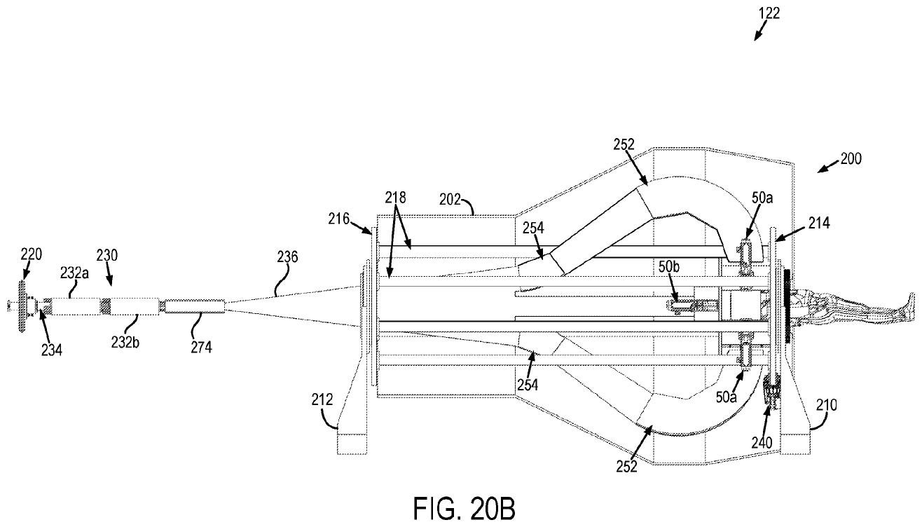

FIGS. 20A and 20B show an example charged particle therapy system 100 implementing a gantry 200 configured as having beam transport magnets 252, 254 arranged in different radial planes that are azimuthally distributed about the longitudinal axis 206 of the rotatable gantry 200. In this example, the gantry 200 is configured as a K0 gantry.

As described above, the beam transport magnets 252, 254 may be, for example, superconducting magnet coils. In the illustrated embodiment, the beam transport magnets 252, 254 include a first set of beam transport magnets 252 configured as focusing magnets and a second set of beam transport 254 magnets configured as defocusing magnets, similar to some embodiments described above. The first set of beam transport magnets 252 can include a two pairs of magnets that are arranged on opposing sides of the longitudinal axis 206. In the illustrated embodiments, the beam transport magnets enable two different azimuthal angles to be selected for the treatment beam trajectory. The gantry 200 may then be rotated about the longitudinal axis 206 (i.e., the rotational axis) to enable the treatment beam trajectory to be moved to different azimuthal angles about the patient.

Alternatively, the first set of beam transport magnets 252 may only include a single pair of magnets arranged on opposing sides of the longitudinal axis 206, and the second set of beam transport magnets 254 may also only include a single pair of magnets arranged on opposing sides of the longitudinal axis 206. In some embodiments, the beam transport assembly 250 may include only a single first beam transport magnet 252 and a single second beam transport magnet 254. In other embodiments, the beam transport assembly may include multiple sets of beam transport magnets 252, 254, which may be arranged about the longitudinal axis 206 at different angles, so as to provide different azimuthal angles along which the treatment beam trajectory may be delivered without having to rotate the gantry 200.

The gantry 200 is supported by a front base 210 and a rear base 212. A front support disc 214 couples the gantry 200 to the front base 210 and a rear support disc 216 couples the gantry 200 to the rear base 212. The front support disc 214 and rear support disc 216 can include bearings, or the like, to rotatably couple the gantry 200 to the front base 210 and the rear base 212, respectively. A plurality of frame tubes 218, or other structural members, couple the front support disc 214 to the rear support disc 216, providing structural support for the gantry 200 (e.g., support for the beam transport magnets 252, 254). The gantry housing 202 is also coupled to or otherwise supported by the front support disc 214 and rear support disc 216. In some embodiments, the gantry housing 202 may also be referred to as a shroud, and may provide radiation shielding. As illustrated in FIG. 20A, a brake 240 can be mounted on the front base 210 and operatively engaged with the front support disc 214, such that when operated the brake 240 can slow and/or stop the rotation of the gantry 200. As a non-limiting example, the brake 240 can include a caliper disc brake, such as a Kobelt 5022-SA spring applied air released caliper disc brake (Kobelt Manufacturing Co. Ltd.; British Columbia, Canada).

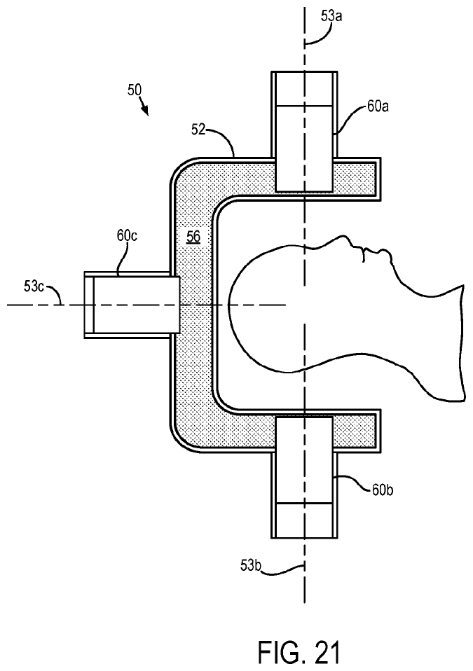

An energy selection system 50 is coupled to the front support disc 214 and surrounds a recessed portion, or bore, that is sized to receive a patient or a portion of the patient's anatomy (e.g., the patient's head). In this example system, the energy selection system 50 includes an azimuthal energy selection system 50a and a vertex energy selection system 50b. As a non-limiting example, as illustrated in FIG. 21, the energy selection system 50 can be constructed with a c-shaped chamber 52, or reservoir, containing a liquid absorber 56.

The volume in different portions of the c-shaped chamber 52 can be adjusted to vary the thickness of liquid absorber 56 along a beam path 53 (e.g., beam path 53a, 53b, and/or 53c) through the respective portion of the chamber 52. In general, adjusting the volume of liquid absorber 56 in one portion of the chamber 52 can also include adjusting the volume of liquid absorber in an adjacent, fluidically coupled portion of the chamber 52. For instance, the volume of liquid absorber 56 may be reduced in one or more portions of the chamber 52 (e.g., along beam paths 53a, 53b) while the volume of liquid absorber 56 may be increased in another portion of the chamber 52 (e.g., along beam path 53c). For instance, the energy selection system 50 can adjust the volume of liquid absorber 56 along the various beam paths 53 using a first azimuthal piston 60a, a second azimuthal piston 60b opposite the first azimuthal piston 60a, and a vertex piston 60c that is oriented along the longitudinal axis 206 of the gantry 200. Linear actuators or other motors can be coupled to the energy selection system 50 and used to translate the pistons 60a, 60b, 60c along the respective beam paths 53a, 53b, 53c under the control of the ESS controller 66.

As described above, the energy selection system 50 is rotatable about the longitudinal axis 206. The energy selection system 50 can thus be rotatably coupled to the front support disc 214 such that the energy selection system 50 is rotated in concert with the beam transport magnets 252, 254. In this way, the ion beam exiting the beam transport magnets 252 remains aligned with the azimuthal energy selection system 50a (e.g., aligned with the azimuthal pistons 60a, 60b).

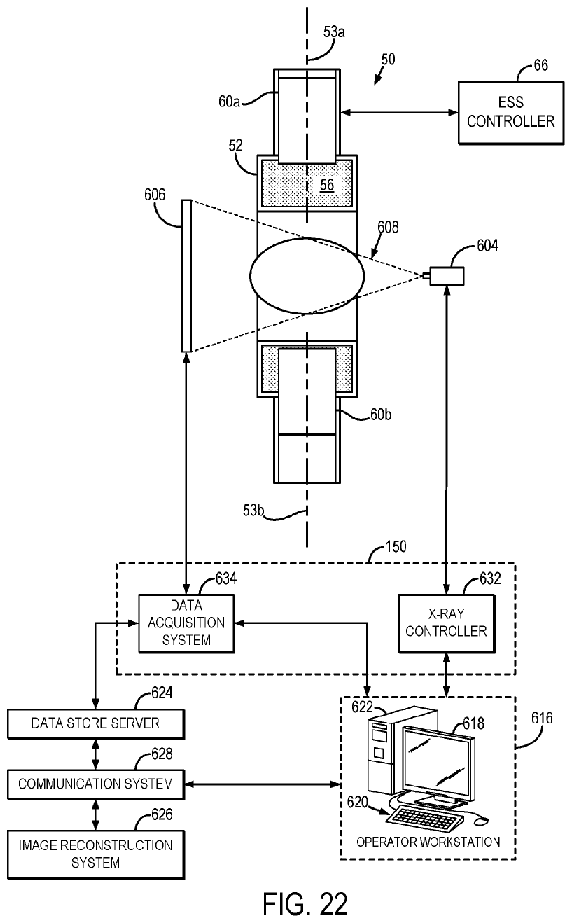

Advantageously, the c-shaped chamber 52 of the energy selection system 50 illustrated in FIG. 21 allows for an x-ray imaging system to be integrated within the gantry 200 of the beam delivery device 122. For example, as shown in FIG. 22, at least one x-ray source 604 can be coupled to the gantry 200 (e.g., coupled to or mounted on the frame tubes 218, or the like). The x-ray source 604 projects an x-ray beam 606, which may be a fan-beam or cone-beam of x-rays, towards a detector array 608 on the opposite side of the energy selection system 50. The detector array 608 includes a number of x-ray detector elements. Together, the x-ray detector elements sense the projected x-rays 606 that pass through a patient that is positioned in the receiving portion of the beam delivery device 122 of the charged particle therapy system 100. Each x-ray detector element produces an electrical signal that may represent the intensity of an impinging x-ray beam and, hence, the attenuation of the beam as it passes through the patient. In some configurations, each x-ray detector is capable of counting the number of x-ray photons that impinge upon the detector. During a scan to acquire x-ray projection data, the gantry 200 and the components mounted thereon rotate about the longitudinal axis 206.

The x-ray imaging system also includes an operator workstation 616, which typically includes a display 618; one or more input devices 620, such as a keyboard and mouse; and a computer processor 622. The computer processor 622 may include a commercially available programmable machine running a commercially available operating system. The operator workstation 616 provides the operator interface that enables scanning control parameters to be entered into the x-ray imaging system (e.g., via the imaging controller 150). In general, the operator workstation 616 is in communication with a data store server 624 and an image reconstruction system 626. By way of example, the operator workstation 616, data store sever 624, and image reconstruction system 626 may be connected via a communication system 628, which may include any suitable network connection, whether wired, wireless, or a combination of both. As an example, the communication system 628 may include both proprietary or dedicated networks, as well as open networks, such as the internet.

The operator workstation 616 is also in communication with the imaging controller 150 that controls operation of the x-ray imaging system. The imaging controller 150 generally includes an x-ray controller 632 and a data acquisition system 634. The x-ray controller 632 provides power and timing signals to the x-ray source 604. The gantry controller 142 of the charged particle therapy system 100 can control the rotational speed and position of the gantry 200 and, by extension, the x-ray source 604.

The data acquisition system 634 samples data from the detector elements of the detector array 608 and converts the data to digital signals for subsequent processing. For instance, digitized x-ray data is communicated from the data acquisition system 634 to the data store server 624. The image reconstruction system 626 then retrieves the x-ray data from the data store server 624 and reconstructs an image therefrom. The image reconstruction system 626 may include a commercially available computer processor, or may be a highly parallel computer architecture, such as a system that includes multiple-core processors and massively parallel, high-density computing devices. Optionally, image reconstruction can also be performed on the processor 622 in the operator workstation 616. Reconstructed images can then be communicated back to the data store server 624 for storage or to the operator workstation 616 to be displayed to the operator or clinician.

Referring again to FIGS. 20A and 20B, the beam delivery device 122 additionally includes a scanning magnet assembly 274 that is located external to the gantry housing 202. Like other components of the beam delivery device 122, the scanning magnet assembly 274 is rotatable.

In the illustrated embodiment, the scanning magnet assembly 274 is located proximal to the rear support disc 216 (i.e., the scanning magnet assembly 274 is arranged between the rear support disc 216 of the gantry 200 and the particle beam generating system 102 (not shown in FIGS. 20A and 20B)). The scanning magnet assembly 274 can include one or more scanning magnets. For example, the scanning magnet assembly 274 can include scanning magnets 270, 272, which can be configured as an X-Y-X scanning magnet, as shown in FIGS. 16A and 16B. Alternatively, the scanning magnet assembly 274 can include scanning magnets 270, 272 that are configured as an SCMZ scanning magnet that scans the exit point of the ion beam in the z-direction and an SCMTH scanning magnet that scans the ion beam in the vertical direction, which may be in the azimuthal direction on the patient.

Advantageously, when the scanning magnet assembly 274 is rotatable with the gantry 200, a simpler scanning magnet configuration can be implemented. For example, the rotatable scanning magnet assembly 274 can, in some examples, include only the SCMZ scanning magnet. By allowing for the scanning magnet assembly 274 to use only an SCMZ scanning magnet, the gap in the gantry 200 can be narrowed, which further simplifies the design, and reduces the overall cost, of the charged particle therapy system 100.

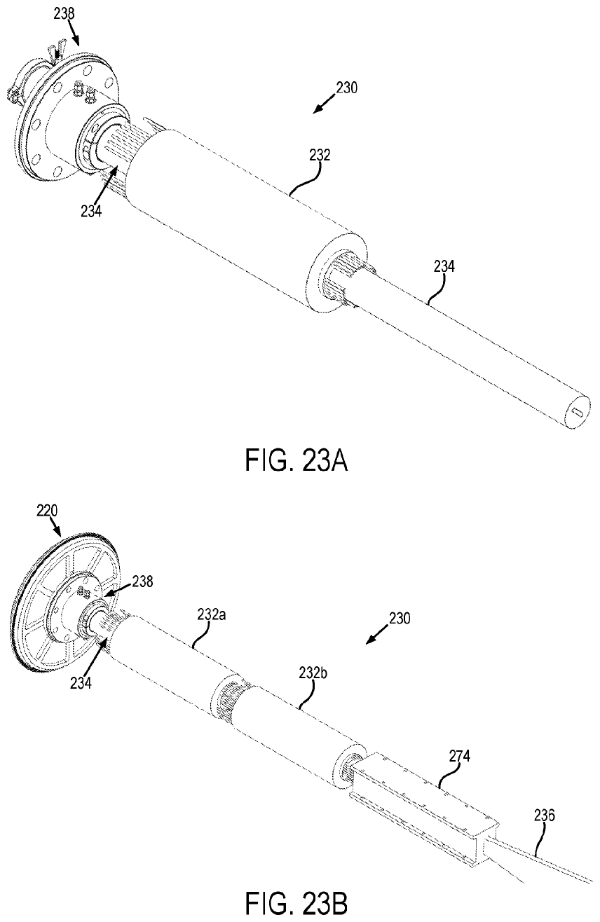

The rotating devices associated with the gantry, including but not limited to the superconducting magnets and their corresponding cryocooler(s), require electrical connections for power and readout. To enable the electrical connections to allow for continuous motion, a rotatable electrical connection 230 is provided, as illustrated in FIGS. 23A and 23B. In the illustrated embodiment, power is supplied to the scanning magnet assembly 274, the beam transport magnets 252, 254, and other electrical components housed within the gantry housing 202 via the rotatable electrical connection 230. As a non-limiting example, the rotatable electrical connection 230 can include one or more slip rings 232. In some embodiments, the rotatable electrical connection 230 includes a single slip ring 232 (e.g., as shown in FIG. 23A). Alternatively, the rotatable electrical connection 230 can include a first slip ring 232a and a second slip ring 232b (e.g., as shown in FIGS. 20A and 20B, and also in FIG. 23B).

The slip ring(s) 232 can be through bore slip rings, which include a central bore. A dynamic beam pipe 234 extends through the central bore of the slip ring(s) 232 and couples to the scanning magnet assembly 274. In operation, the ion beam is generated by the particle beam generating system 102 and delivered through the dynamic beam pipe 234 towards the scanning magnet assembly 274. The ion beam then travels through the scanning magnetic assembly 274 where it is scanned in one or more spatial directions (e.g., horizontal and vertical, radial and azimuthal) by the scanning magnets 270, 272, as described above in more detail. A flange mount 238 can be provided at the proximal end of the rotatable electrical connection 230, which in some embodiments can coupled to a labyrinth face seal 220 for providing a rotatable fluid coupling for cryogen (as shown in FIG. 23B and described in more detail below).

In some configurations, a scanner beam pipe 236 is coupled to the scanning magnet assembly 274 at the distal end of the scanning magnet assembly 274 (i.e., at the end of the scanning magnet assembly 274 where the ion beam exits the scanning magnet assembly 274 towards the gantry 200). The scanner beam pipe 236 extends from the distal end of the scanning magnet assembly 274 towards the distal end of the gantry 200 where the ion beam is delivered to the patient. In the illustrated embodiment, the scanner beam pipe 236 includes three branches: a first branch 236a that extends toward the first azimuthal energy selection system 50a, a second branch that extends toward the second azimuthal energy selection system 50a, and a third branch that extends along the longitudinal axis 206 towards a vertex energy selection system 50b. The first and second branches of the scanner beam pipe 236 pass through first and second sets of beam transport magnets 252, 254. For example, the scanner beam pipe 236 can be arranged between pairs of beam transport magnets (e.g., the first branch of the scanner beam pipe 236 can be arranged between a first pair of beam transport magnets in the first set of beam transport magnets 252 and also between a first pair of beam transport magnets in the second set of beam transport magnets 254; the second branch of the scanner beam pipe 236 can be arranged between a second pair of beam transport magnets in the first set of beam transport magnets 252 and also between a second pair of beam transport magnets in the second set of beam transport magnets 254). The second set of beam transport magnets 254 can be proximal to the first set of beam transport magnets 252, similar to the configurations described above. In the illustrated embodiment, the second set of beam transport magnets 254 can be configured as defocusing magnets providing an approximately 45 degree bend, and the first set of beam transport magnets 252 can be configured as focusing magnets providing an approximately 135 degree bend.

In general, the scanner beam pipe 236 can be constructed as a hollow structure though which the ion beam is transported from the scanning magnet assembly 274 towards the patient. For instance, the scanner beam pipe 236 can include a vacuum chamber. The scanner beam pipe 236 may be composed of a material such as stainless steel, and in some configurations can be constructed as a pipe with a thin slit.

Superconducting magnets, such as the beam transport magnets 252, 254, typically require a cryocooler with the cold head mounted to the magnet. The cryocooler gas line is connected to a compressor. The compressor is typically not mounted on the rotating gantry 200 and so the gas line is rotated as the gantry 200 rotates about the patient. To facilitate the continuous motion, a rotary coupling is provided using a labyrinth face seal 220 to allow the cryogen transport between the non-rotating compressor and the rotating cryocooler housed within the gantry housing 202.

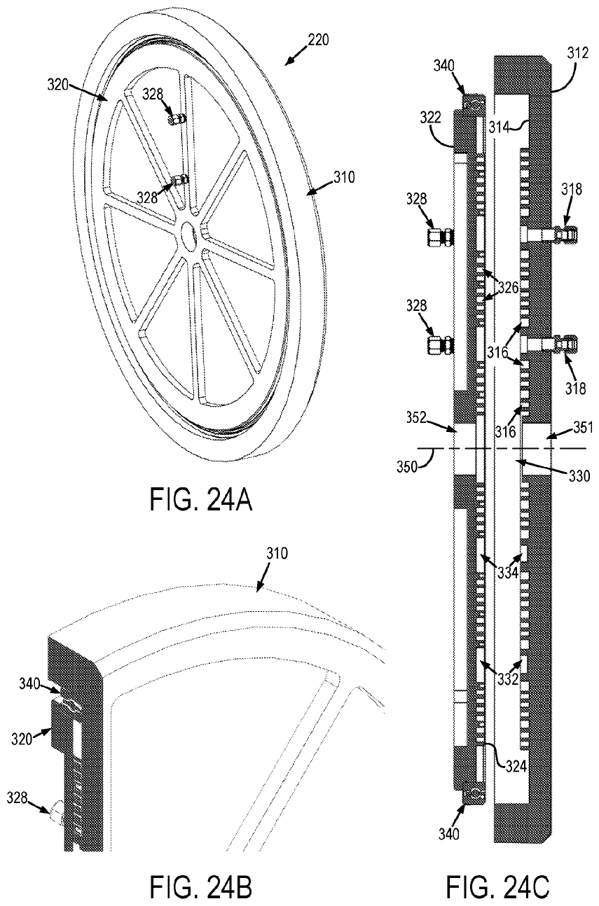

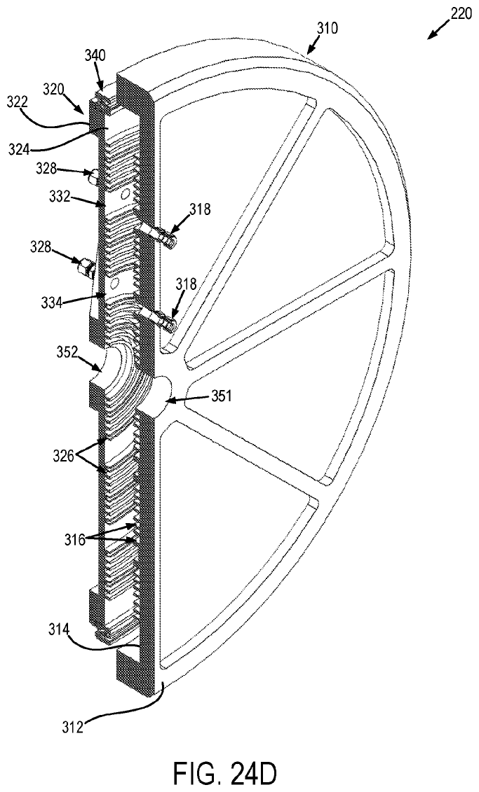

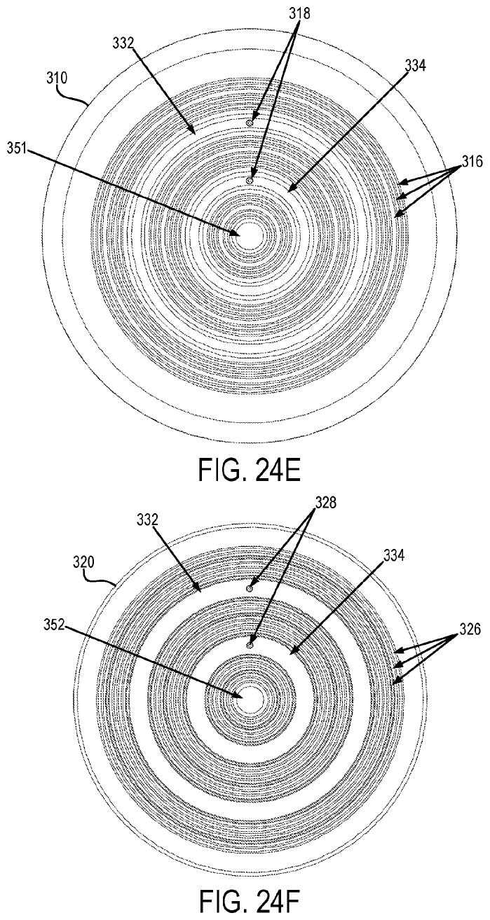

FIGS. 24A-24F illustrate an example rotatable fluid coupling constructed as a labyrinth face seal 220 that fluidically couples a compressor to the gantry housing 202 to one or more cryocoolers housed within the gantry housing 202. The labyrinth face seal 220 provides cryogen (e.g., helium gas or other suitable liquid or gaseous cryogens) from the compressor to the cryocooler(s). In some embodiments, the compressor is located external to the gantry housing 202 and does not rotate with the gantry 200, but the cryocooler is located within the gantry housing 202 and therefore does rotate with the gantry 200. In these instances, the labyrinth face seal 220 provides a rotatable fluid coupling between the compressor and the cryocooler.

In general, the labyrinth face seal is composed of a fixed face plate 310 having an external side 312 and an internal side 314, and a rotatory face plate 320 having an external side 322 and an internal side 324 that faces and mates with the internal side 314 of the fixed face plate 310. The fixed face plate 310 and the rotary face plate 320 can be both be constructed as coaxially aligned circular annular discs. The internal side 314 of the fixed face plate 310 has a plurality of grooves 316 formed therein, and the internal side 324 of the rotary face plate 320 has a matching plurality of grooves 326 formed therein, which interlock with the plurality of grooves 316 formed in the internal side 314 of the fixed face plate 310.

The plurality of grooves 316, 326 operatively engage with each other (e.g., interlock with each other) and provide a track for the rotary face plate 320 to rotate about the rotational axis 350. The grooves 316, 326 also prevent or otherwise reduce leakage of cryogen (e.g., leakage out of the labyrinth face seal 220, leakage between the first flow path 332 and the second flow path 334, etc.). In some alternative configurations, grooves may be formed only in the internal side 314 of the fixed face plate 310 (e.g., grooves 316), or only in the internal side 324 of the rotary face plate 320 (e.g., grooves 326). In the illustrated embodiment, the grooves 326 formed in the internal side 324 of the rotary face plate 320 include a plurality of grooves of increasing diameter extending outward from the central bore 352 of the rotary face plate 320. Similarly, the grooves 316 formed in the internal side 314 of the fixed face plate 310 include a plurality of grooves of increasing diameter extending outward from the central bore 351 of the fixed face plate.

A ball bearing 340 is arranged around the circumference of the rotary face plate 320 and facilitates rotation of the rotary face plate 320 about the rotational axis 350 relative to the fixed face plate 310. For example, the ball bearing 340 may include an inner ring coupled to the circumference of the rotary face plate 320 and an outer ring coupled to an inner circumferential surface of the fixed face plate 310. A plurality of rollers, or balls, is arranged between the inner ring and the outer ring to facilitate rotation of the rotary face frame 320.

Cryogen (e.g., liquid cryogen, gaseous cryogen) is delivered to and from the interior space 330 of the labyrinth face seal 220 via one or more ports 318 on the external side 312 of the fixed face plate 310. The one or more ports 318 may be, for example, an instrumentation fitting, (e.g., a Yor-Lok fitting) to which stainless steel tubing or other tubing can be fluidically coupled. Likewise, the cryogen can be delivered to and from the interior space 330 of the labyrinth face seal 220 via one or more ports 328 on the external side 322 of the rotary face plate 320. The one or more ports 328 may be, for example, an instrumentation fitting, (e.g., a Yor-Lok fitting) to which stainless steel tubing or other can be fluidically coupled.

When mated together, the fixed face plate 310 and the rotary face plate 320 define an interior volume 330, or space, through which cryogen is able to flow from one side of the labyrinth face seal 220 to the other side (e.g., from the external side 312 of the fixed face plate 310 to the external side 322 of the rotary face plate 320). In particular, a first flow path 332 is defined as a circular channel formed in the internal side 314 of the fixed face plate 310 and a matching circular channel formed in the internal side 324 of the rotary face plate 320. Cryogen can be provided to and from the first flow path 332 via the ports 318, 328. Similarly, a second flow path 334 is defined as a circular channel formed in the internal side 314 of the fixed face plate 310 and a matching circular channel formed in the internal side 324 of the rotary face plate 320. Cryogen can be provided to and from the second flow path 334 via another pair of the ports 318, 328. The first flow path 332 and the second flow path 334 are defined as circular channels having different diameters. For example, in the illustrated embodiment the first flow path 332 is a circular channel having a larger diameter than the circular channel defining the second flow path 334.

In operation, cryogen can circulate from the compressor to the labyrinth face seal 220 via one of the ports 318 on the external side 312 of the fixed face seal 310 (e.g., an inlet port), into the interior volume of the labyrinth face seal 220, and to the cryocooler via one of the ports 328 on the external side 322 of the rotary face seal 320 (e.g., an outlet port). Cryogen can also circulate from the cryocooler to the labyrinth face seal 220 via one of the ports 328 on the external side 322 of the rotary face seal 320 (e.g., an inlet port), into the interior volume 330 of the labyrinth face seal 220, and to the compressor via one of the ports 318 on the external side 312 of the fixed face seal 310 (e.g., an outlet port). In some configurations, the labyrinth face seal 220 can include the first flow path 332 for cryogen flowing from the compressor to the cryocooler and the second flow path 334 for cryogen flowing from the cryocooler to the compressor.

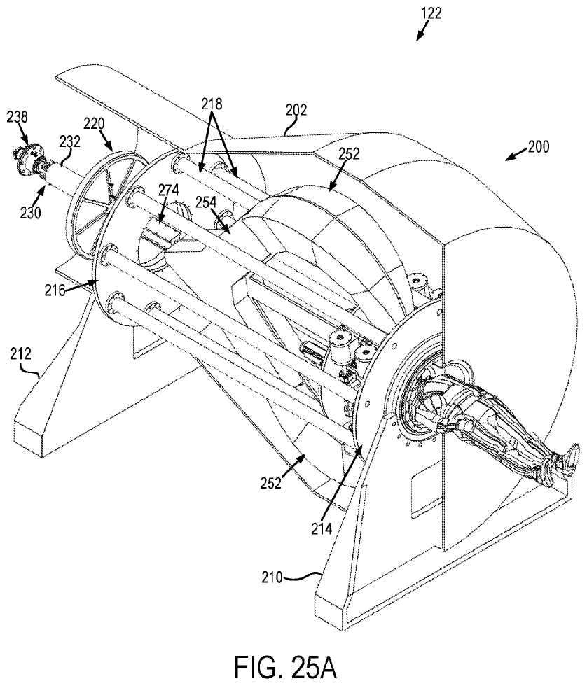

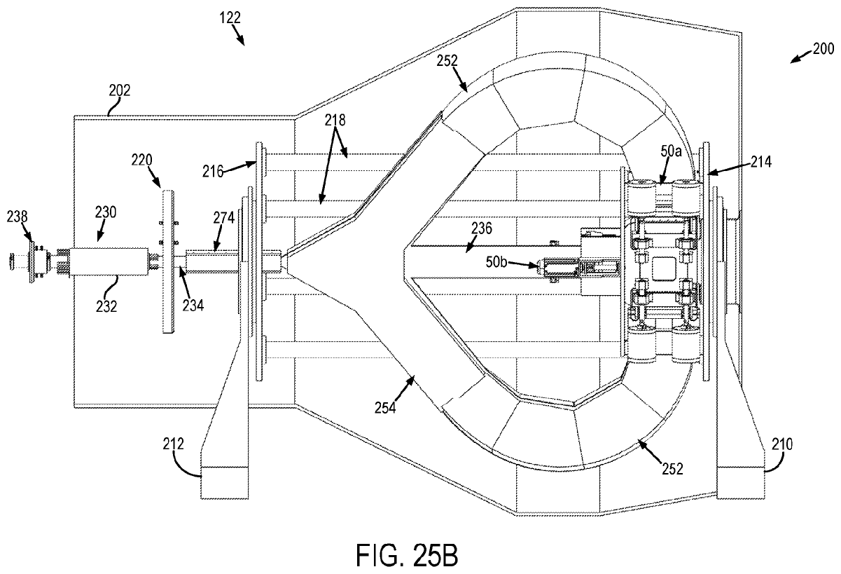

FIGS. 25A and 25B show an example charged particle therapy system 100 implementing a gantry 200 configured as a divergent KN gantry having a more compact form than the example shown in FIGS. 20A and 20B. In the illustrated embodiment, the height of the gantry 200 is approximately 3 meters, the width of the gantry 200 is approximately 3 meters (e.g., the outer diameter of the gantry 200 is approximately 3 meters), and the length of the gantry 200 is approximately 4 meters.

Like the charged particle therapy system 100 illustrated in FIGS. 20A and 20B, the divergent KN gantry system shown in FIGS. 25A and 25B includes beam transport magnets 252, 254 arranged in different radial planes that are azimuthally distributed about the longitudinal axis 206 of the rotatable gantry 200. As described above, the beam transport magnets 252, 254 may be, for example, superconducting magnet coils. In the illustrated embodiment, the beam transport magnets 252, 254 include a first set of beam transport magnets 252 configured as focusing magnets and a second set of beam transport 254 magnets configured as defocusing magnets, as described above. The first set of beam transport magnets 252 can include a two pairs of magnets that are arranged on opposing sides of the longitudinal axis 206. In the illustrated embodiments, the beam transport magnets enable two different azimuthal angles to be selected for the treatment beam trajectory. The gantry 200 may then be rotated about the longitudinal axis 206 (i.e., the rotational axis) to enable the treatment beam trajectory to be moved to different azimuthal angles about the patient.

Alternatively, the first set of beam transport magnets 252 may only include a single pair of magnets arranged on opposing sides of the longitudinal axis 206, and the second set of beam transport magnets 254 may also only include a single pair of magnets arranged on opposing sides of the longitudinal axis 206. In some embodiments, the beam transport assembly 250 may include only a single first beam transport magnet 252 and a single second beam transport magnet 254. In other embodiments, the beam transport assembly may include multiple sets of beam transport magnets 252, 254, which may be arranged about the longitudinal axis 206 at different angles, so as to provide different azimuthal angles along which the treatment beam trajectory may be delivered without having to rotate the gantry 200.

The gantry 200 is supported by a front base 210 and a rear base 212. A front support disc 214 couples the gantry 200 to the front base 210 and a rear support disc 216 couples the gantry 200 to the rear base 212. The front support disc 214 and rear support disc 216 can include bearings, or the like, to rotatably couple the gantry 200 to the front base 210 and the rear base 212, respectively. A plurality of frame tubes 218, or other structural members, couple the front support disc 214 to the rear support disc 216, providing structural support for the gantry 200 (e.g., support for the beam transport magnets 252, 254). The gantry housing 202 is also coupled to or otherwise supported by the front support disc 214 and rear support disc 216. In some embodiments, the gantry housing 202 may also be referred to as a shroud, and may provide radiation shielding.

In the illustrated embodiment, the gantry 200 and gantry housing 202 are more compact than in the system illustrated in FIGS. 20A and 20B. Here, the labyrinth face seal 220 that provides the rotatable fluid coupling for the cryogen supplying the beam transport magnets 252, 254; the rotatable electrical connection 230; and the scanning magnet assembly 274 are housed within the gantry housing 202. Advantageously, as described above, the c-shaped chamber 52 of the energy selection system 50 illustrated in FIG. 21 allows for an x-ray imaging system to be integrated within the gantry 200 of the beam delivery device 122. This allows for imaging of the patient during the delivery of charged particle therapy (e.g., in snaps between ion beam delivery and/or before or after treatment).

Similar to the beam delivery device 122 illustrated in FIGS. 20A and 20B, the divergent KN gantry system shown in FIGS. 25A and 25B can include an energy selection system 50 that is coupled to the front support disc 214 and that surrounds a recessed portion, or bore, that is sized to receive a patient or a portion of the patient's anatomy (e.g., the patient's head). The energy selection system 50 can include an azimuthal energy selection system 50a defining two different azimuthal beam paths, and a vertex energy selection system 50b. As a non-limiting example, as illustrated in FIG. 21, the energy selection system 50 can be constructed with a c-shaped chamber 52, or reservoir, containing a liquid absorber 56.

The present disclosure has described one or more preferred embodiments, and it should be appreciated that many equivalents, alternatives, variations, and modifications, aside from those expressly stated, are possible and within the scope of the invention.

1. A charged particle beam delivery assembly for use in a charged particle therapy system, comprising:
an annular gantry having a bore extending along a longitudinal axis; and
a plurality of beam transport magnets coupled to the gantry, each beam transport magnet extending within a plane oriented relative to the longitudinal axis such that a charged particle beam entering the annular gantry along a first beam trajectory at an entrance point is bent towards the longitudinal axis by one or more magnetic fields generated by the beam transport magnets, thereby exiting the annular gantry at an exit point along a second beam trajectory.

2. The beam delivery assembly of claim 1, wherein each of the beam transport magnets extends in a radial plane oriented at an azimuthal angle with respect to the longitudinal axis of the gantry.

3. The beam delivery assembly of claim 2, wherein the beam transport magnets are configured to generate the one or more magnetic fields such that a charged particle beam entering the gantry at the entrance point along a direction oriented along the longitudinal axis is bent towards the longitudinal axis.

4. The beam delivery assembly of claim 2, further comprising a scanning magnet that generates a magnetic field that steers the charged particle beam in a vertical direction thereby adjusting a longitudinal position of the exit point of the charged particle beam as it exits the gantry.

5. The beam delivery assembly of claim 2, further comprising a scanning magnet that generates a magnetic field that steers the charged particle beam in a horizontal direction thereby adjusting an azimuthal position of the exit point of the charged particle beam as it exits the gantry.

6. The beam delivery assembly of claim 2, further comprising:
a first scanning magnet that generates a magnetic field that steers the charged particle in a vertical direction thereby adjusting a longitudinal position of the exit point of the charged particle beam as it exits the gantry; and
a second scanning magnet that generates a magnetic field that steers the charged particle beam in a horizontal direction thereby adjusting an azimuthal position of the exit point of the charged particle beam as it exits the gantry.

7. The beam delivery assembly of claim 6, wherein the first scanning magnet comprises a first scanning magnet part and a second scanning magnet part, wherein the first scanning magnet part is arranged on a first side of the second scanning magnet and the second scanning magnet part is arranged on a second side of the second scanning magnet opposite the first side along the longitudinal axis.

8. The beam delivery assembly of claim 7, wherein the second scanning magnet is centered between the first scanning magnet part and the second scanning magnet part, such that the first scanning magnet and the second scanning magnet have a common virtual source position.

9. The beam delivery assembly of claim 1, wherein the plurality of beam transport magnets define a plurality of different azimuthally distributed radial sectors of the annular gantry, each radial sector defining a different azimuthal angle along which the charged particle beam can be steered towards the longitudinal axis.

10. The beam delivery assembly of claim 1, wherein each of the beam transport magnets extends in an axial plane and are circumferentially distributed about the longitudinal axis of the gantry.

11. The beam delivery assembly of claim 10, wherein the beam transport magnets are configured to generate the one or more magnetic fields such that a charged particle beam entering the gantry at the entrance point along a direction oriented towards the longitudinal axis is steered towards the longitudinal axis.

12. The beam delivery assembly of claim 11, further comprising a scanning magnet that generates a magnetic field that steers the charged particle beam in a horizontal direction thereby adjusting a longitudinal position of the exit point of the charged particle beam as it exits the gantry.

13. The beam delivery assembly of claim 11, further comprising a scanning magnet that generates a magnetic field that steers the charged particle beam in a vertical direction thereby adjusting an azimuthal position of the exit point of the charged particle beam as it exits the gantry.

14. The beam delivery assembly of claim 11, further comprising:
a first scanning magnet that generates a magnetic field that steers the charged particle beam in a horizontal direction thereby adjusting a longitudinal position of the exit point of the charged particle beam as it exits the gantry; and
a second scanning magnet that generates a magnetic field that steers the charged particle beam in a vertical direction thereby adjusting an azimuthal position of the exit point of the charged particle beam as it exits the gantry.

15. The beam delivery assembly of claim 14, wherein the second scanning magnet comprises a first scanning magnet part and a second scanning magnet part, wherein the first scanning magnet part is arranged on a first side of the first scanning magnet and the second scanning magnet part is arranged on a second side of the first scanning magnet opposite the first side along the longitudinal axis.

16. The beam delivery assembly of claim 15, wherein the first scanning magnet is centered between the first scanning magnet part and the second scanning magnet part, such that the first scanning magnet and the second scanning magnet have a common virtual source position.

17. The beam delivery assembly of claim 1, wherein the beam transport magnets are configured to generate one or more magnetic field gradients within the annular gantry, such that the one or more magnetic field gradient further steer the charged particle beam from the first beam trajectory to the second beam trajectory.

18. The beam delivery assembly of claim 17, wherein the one or more magnetic field gradients comprise a radial magnetic field gradient.

19. The beam delivery assembly of claim 17, wherein the one or more magnetic field gradients comprise an axial magnetic field gradient.

20. The beam delivery assembly of claim 1, wherein the annular gantry is a static annular gantry that holds the plurality of beam transport magnetic in a fixed position about the longitudinal axis.

21. The beam delivery assembly of claim 1, wherein the plurality of beam transport magnets fully encircle the bore of the annular gantry.

22. The beam delivery assembly of claim 1, wherein the plurality of beam transport magnets span a range of azimuthal angles about the bore of the annular gantry that is less than 360 degrees.

23. The beam delivery assembly of claim 22, wherein the plurality of beam transport magnets comprise a beam transport magnet module that is rotatable about the longitudinal axis.

24. The beam delivery assembly of claim 22, wherein the plurality of beam transport magnets comprises two beam transport magnets each extending in a different radial plane oriented at a different azimuthal angle with respect to the longitudinal axis of the gantry.

25. The beam delivery assembly of claim 22, wherein the plurality of beam transport magnets comprises three beam transport magnets each extending in a different radial plane oriented at a different azimuthal angle with respect to the longitudinal axis of the gantry.

26. The beam delivery assembly of claim 1, wherein the annular gantry has a diameter in a range of 10 feet to 16 feet.

27. The beam delivery assembly of claims 1- or 26, wherein the annular gantry has a length in a range of 4 feet to 15 feet.

28. The beam delivery assembly of 1, wherein the bore comprises a recessed region that partially extends into a housing of the gantry to define a region sized to receive a portion of a patient's anatomy.

29-79. (canceled)
